(19) United States
(12) Patent Application Publication
(10) Pub. No.: US 2014/0106009 A1
(43) Pub. Date: Apr. 17, 2014

(54) METHODS AND COMPOSITIONS FOR MAINTAINING AND IMPROVING THE HEALTH OF SKIN

(71) Applicant: Graham Matheson, Cronulla (AU)

(72) Inventor: Graham Matheson, Cronulla (AU)

(73) Assignee: CIMTECH Pty Limited, Newcastle West, New South Wales (AU)

(21) Appl. No.: 13/883,196

(22) PCT Filed: Nov. 3, 2011
(86) PCT No.: PCT/AU2011/001410

§ 371 Date: Dec. 23, 2013

(30) Foreign Application Priority Data
Nov. 3, 2010 (AU) 2010904905

(51) Int. Cl. A61K 8/97; A61Q 19/00; A61Q 19/02; A61Q 19/08

(52) U.S. Cl. CPC A61K 8/97; A61Q 19/08; A61Q 19/00; A61Q 19/02; A61Q 19/007
USPC 424/727

(57) ABSTRACT

Biologically active extracts of one or more of Vigna sp., Terminalia sp or Cocos nucifera and compositions comprising one or more of the extracts for the maintenance and improvement of the health and/or appearance of the skin and/or an epidermal appendage are described.

### FIELD OF THE INVENTION

The present invention relates generally to the use of compositions comprising plant extracts for the maintenance and improvement of the health and/or appearance of the skin and/or an epidermal appendage.

### BACKGROUND OF THE INVENTION

Human skin is the outermost barrier of the human body, protecting against external detrimental influences. After birth skin matures for about another year to its full protective potential and is then the largest metabolically active organ of the human body. As a person ages the skin begins to change in an age-related manner, caused by a variety of influences and leading to a number of consequences, ranging from aesthetically unpleasing to clinically relevant conditions.

Visible signs of skin aging include the appearance of fine lines and wrinkles, dullness and lack of skin radiance; uneven skin tone; dry, sensitive and itchy skin; age spots; blotchiness, broken capillaries, intracutaneous and subcutaneous bleeding; a decrease in elasticity and an increase skin sagging; an increase in skin roughness and the appearance of visible pores.

The use of cosmetic compositions is increasing worldwide with both men and women wishing to improve the visual appearance of their skin including decreasing the visible signs of aging. Accordingly, there is much interest in compositions and methods for maintaining and improving the health of the skin.

### SUMMARY OF THE INVENTION

According to a first aspect of the present invention there is provided a composition comprising a biologically active extract of one or more of Vigna sp., Terminalia sp., or Cocos nucifera for maintaining and improving of the health and/or appearance of the skin and/or an epidermal appendage. The composition may further comprise a biologically active extract of Hibiscus sp. The improvement may be, but is not limited to, a reduction in the appearance of fine line and wrinkles, an increase in skin radiance, an increase in skin tone, an increase in skin texture and/or firmness, a reduction in acne scars, a decrease in age spots, a reduction in the blotchiness, broken capillaries, intracutaneous and/or subcutaneous bleeding of the skin, an increase in the lightness of skin and/or an epidermal appendage, a reduction of visible pores, a reduction in stretch marks, an increase in skin elasticity and/or a reduction in skin sagging, a reduction in skin sensitivity, a reduction in skin roughness, restoration of the physiological balance of the skin and/or an epidermal appendage, an increase in trans-epidermal water loss, or an increase in the natural moisturising factors in the skin.

In one embodiment the composition is applied to aging or aged skin. The aging or aged skin may have age related skin atrophy such as associated with oestrogen deficiency.

In one embodiment the Vigna sp. may be Vigna marina (Burm.) Merr., the Terminalia sp. may be Terminalia catappa L. and the Hibiscus sp. may be Hibiscus tiliaceus L.

In another embodiment the composition comprises an extract of Vigna marina (Burm.) Merr. The extract may be derived from a combination of Vigna marina (Burm.) Merr. and Terminalia catappa L., a combination of Vigna marina (Burm.) Merr. and Cocos nucifera L., or a combination of Vigna marina (Burm.) Merr., Cocos nucifera L., and Terminalia catappa L. The extract may be derived from a combination of Vigna marina (Burm.) Merr., Cocos nucifera L., Terminalia catappa L. and Hibiscus tiliaceus L.

The extract may be one or more of a leaf, root, bark, wood, stem, fruit, shoot, seed, seed capsule, vine, bean, flower husk or nut extract. Where derived from Vigna marina (Burm.) Merr., Terminalia catappa L., and Hibiscus tiliaceus L. typically the extract is a leaf, vine, husk and/or bean extract. Where derived from Cocos nucifera typically the extract is a husk and/or nut extract.

The extract may be prepared using, and/or comprises, a lipid, a plant based oil, a hydrocarbon and/or an alcohol. The plant based oil may be derived from, for example, seeds or fruit. In a particular embodiment the extract is prepared using, and/or comprises, Cocos nucifera oil. The Cocos nucifera oil may be virgin Cocos nucifera oil, refined Cocos nucifera oil; hydrogenated Cocos nucifera oil or fractionated Cocos nucifera oil.

The composition may further comprise a cosmetically or pharmaceutically acceptable carrier, diluent and/or excipient.

Typically the composition is applied topically to the skin or to an epidermal appendage of a subject. Epidermal appendages include, but are not limited to, hair, hair follicles, nails, cuticles, and teeth.

A second aspect of the invention provides a method for maintaining and improving the health and/or appearance of the skin and/or an epidermal appendage, the method comprising applying to the skin and/or epidermal appendage of a subject a composition comprising a biologically active extract of one or more of Vigna sp., Terminalia sp. or Cocos nucifera. The composition may further comprise a biologically active extract of Hibiscus sp. The improvement may be but is not limited to a reduction in the appearance of fine line and wrinkles, an increase in skin radiance, an increase in skin tone, an increase in skin texture and/or firmness, a reduction in acne scars, a decrease in age spots, a reduction in the blotchiness, broken capillaries, intracutaneous and/or subcutaneous bleeding of the skin, an increase in the lightness of skin and/or an epidermal appendage, a reduction of visible pores, a reduction in stretch marks, an increase in skin elasticity and/or a reduction in skin sagging, a reduction in skin roughness, a reduction in skin sensitivity, restoration of the physiological balance of the skin and/or an epidermal appendage, an increase in trans-epidermal water loss, or an increase in the natural moisturising factors in the skin.

In one embodiment the composition is applied to aging or aged skin. The aging or aged skin may have age related skin atrophy such as associated with oestrogen deficiency.

In one embodiment the Vigna sp. may be Vigna marina (Burm.) Merr., the Terminalia sp. may be Terminalia catappa L. and the Hibiscus sp. may be Hibiscus tiliaceus L. In another embodiment Cocos nucifera oil may be applied to the skin and/or epidermal appendage prior to or after the composition is applied.

The subject may be a human.

A third aspect of the invention provides a use of a composition comprising a biologically active extract of one or more of Vigna sp., Terminalia sp. or Cocos nucifera for the manufacture of a medicament for maintaining and improving the health and/or appearance of the skin and/or an epidermal appendage. The composition may further comprise a biologically active extract of Hibiscus sp. The improvement may be but is not limited to a reduction in the appearance of fine line and wrinkles, an increase in skin radiance, an increase in skin tone, an increase in skin texture and/or firmness, a reduction in acne scars, a decrease in age spots, a reduction in the blotchiness, broken capillaries, intracutaneous and/or subcutaneous bleeding of the skin, an increase in the lightness of skin and/or an epidermal appendage, a reduction of visible pores, a reduction in stretch marks, an increase in skin elasticity and/or a reduction in skin sagging, a reduction in skin roughness, a reduction in skin sensitivity, restoration of the physiological balance of the skin and/or an epidermal appendage, an increase in trans-epidermal water loss, or an increase in the natural moisturising factors in the skin.

In one embodiment the composition is applied to aging or aged skin. The aging or aged skin may have age related skin atrophy such as associated with oestrogen deficiency.

In one embodiment the Vigna sp. may be Vigna marina (Burm.) Merr., the Terminalia sp. may be Terminalia catappa L. and the Hibiscus sp. may be Hibiscus tiliaceus L. In another embodiment Cocos nucifera oil may be applied to the skin and/or epidermal appendage prior to or after the composition is applied.

### BRIEF DESCRIPTION OF THE DRAWINGS

The present invention will now be described, by way of non-limiting example only, with reference to the accompanying drawings in which:

FIG. 1 shows histologic differences in treated (B, D, F and H) and untreated (A, C, E and G) skin of 18 month old, oestrogen deficient (ovaries removed at age 6 weeks) rats. The treated skin was subjected to a daily application of a combination extract of Vigna marina (Burm.) Merr., Cocos nucifera L., Terminalia catappa L. and Hibiscus tiliaceus L. in coconut oil for 7 days. The skin samples were stained with hematoxylin and eosin and are shown at ×400 magnification.

FIG. 2 shows the epithelial thickness of treated (daily application of a combination extract of Vigna marina (Burm.) Merr., Cocos nucifera L., Terminalia catappa L. and Hibiscus tiliaceus L. in coconut oil) and control (untreated) oestrogen deficient skin following 7 days of treatment (A) and oestrogen deficient skin adjacent to treatment area following 21 days of treatment (B).

FIG. 3 shows the percentage change in epithelial thickness in oestrogen deficient skin following 7 days treatment comprising daily application of a combination extract of Vigna marina (Burm.) Merr., Cocos nucifera L., Terminalia catappa L. and Hibiscus tiliaceus L. Bar 1 (Pre-extraction), the extract is in coconut oil; Bar 2 (Ethanol extract), removal of coconut oil from the post-extraction extract; Bar 3 (Post extraction), the residual coconut oil fraction following ethanol extraction (Bar 2).

FIG. 4 shows histologic profiles of skin of 10 to 14 weeks old rats following daily application for 7 days of A) no treatment; B) coconut oil; C) Vigna marina (Burm.) Merr. in coconut oil, D) Vigna marina (Burm.) Merr. in ethanol; E) Vigna marina (Burm.) Merr. in coconut oil following extraction with ethanol and isopentane; F) Terminalia catappa L. in coconut oil; G) Terminalia catappa L. in ethanol; H) Terminalia catappa L. in hydrocarbon following ethanol extraction; I) Terminalia catappa L. in coconut oil following extraction with ethanol and hydrocarbon; and J) Terminalia catappa L. in coconut oil following extraction with ethanol and hydrocarbon. The skin samples were stained with hematoxylin and eosin and are shown at ×400 magnification.

FIG. 5 shows A) the percentage change in epithelial thickness of skin of 10 to 14 weeks old rats following daily application for 7 days of coconut oil (Coconut Oil), Terminalia catappa L. in ethanol following extraction with ethanol and hydrocarbon (HeK in Ethanol), Terminalia catappa L. in coconut oil following extraction with ethanol and hydrocarbon (HeK in Coconut Oil), and Terminalia catappa L. in coconut oil following extraction with ethanol (eK in Coconut Oil); and B) the epithelial thickness of skin of 10 to 14 weeks old rats following daily application for 7 days of Vigna marina (Burm.) Merr. in coconut oil following extraction with ethanol (eP in Coconut Oil), Vigna marina (Burm.) Merr. in ethanol following extraction with ethanol (eP Control), Vigna marina (Burm.) Merr. in coconut oil following extraction with ethanol and hydrocarbon (HeP in Coconut Oil), and Vigna marina (Burm.) Merr. in ethanol following extraction with ethanol and hydrocarbon (HeP Control).

FIG. 6 shows the percent change in epithelial thickness of skin of 10 to 14 weeks old rats following 7 days of treatment with A) Vigna marina (Burm.) Merr. extract in coconut oil (Plant P); Cocos nucifera L. extract in coconut oil (Plant N); Terminalia catappa L. extract in coconut oil (Plant N); and Hibiscus tiliaceus L. in coconut oil (Plant A) and B) pairwise combination of the aforementioned extracts.

FIG. 7 shows histologic differences in skin of 10 to 14 weeks old rats treated with an extract of Vigna marina (Burm.) Merr., Cocos nucifera L. and Terminalia catappa L. in coconut oil (100%) prepared as described in Example 10, at concentrations of A) 0%; B) 1%; C) 5%; D) 10%; E) 50%; and F) 100%. The skin samples were stained with hematoxylin and eosin and are shown at ×400 magnification.

FIG. 8 shows the epithelial thickness (A) and percent change in epithelial thickness of skin of 10 to 14 weeks old rats treated with a daily application of a combination extract of Vigna marina (Burm.) Merr., Cocos nucifera L. and Terminalia catappa L. in coconut oil (NPK in oil) prepared as described in Example 10, at concentrations of 0%, 1%; 5%; 10%, 25%, 50%, 40% and 100%; (B) of skin of 10 to 14 weeks old rats treated with a daily application of a combination extract of Vigna marina (Burm.) Merr., Cocos nucifera L. and Terminalia catappa L. in coconut oil (NPK in oil) prepared as described in Example 10, at concentrations of 0%, 1%; 5%; 10%, 50%, and 100%.

FIG. 9 shows the histology of skin treated with an extract of Vigna marina (Burm.) Merr., Cocos nucifera L. and Terminalia catappa L. in coconut oil at A) 30% in chlorhexidine; B) 5% in hydroderm moisturiser; C) 25% in chloramphenicol solution; D) 50% in chloramphenicol ointment; and E) 0% in chloramphenicol ointment.

FIG. 10 shows histologic changes in treated (A, C, E and G) and untreated (B, D, F and H) surgically created wound sites on 12 week old healthy female rats at 3, 5, 7 and 10 days post operative. The treated wounds were subjected to a daily application of a combination extract of Vigna marina (Burm.) Merr., Cocos nucifera L., Terminalia catappa L. and Hibiscus tiliaceus L. in coconut oil. The skin samples were stained with hematoxylin and eosin and are shown at x400 magnification.

FIG. 11 shows the increase in mechanical wound strength (A) and the average mechanical wound strength (B) for wounds sites of treated animals (test) and untreated animals (control) over the course of 10 days. The treated wounds were subjected to a daily application of a combination extract of Vigna marina (Burm.) Merr., Cocos nucifera L., Terminalia catappa L. and Hibiscus tiliaceus L. in coconut oil. FIG. 11A shows the linear correlation and FIG. 11B is modelled into a non linear healing curve.

### DETAILED DESCRIPTION OF THE INVENTION

The articles “a” and “an” are used herein to refer to one or to more than one (i.e., to at least one) of the grammatical object of the article. By way of example, “an element” means one element or more than one element.

Throughout this specification and the claims which follow, unless the context requires otherwise, the word “comprise”, and variations such as “comprises” or “comprising”, will be understood to imply the inclusion of a stated integer or step or group of integers or steps but not the exclusion of any other integer or step or group of integers or steps.

As used herein the term “extract” refers to an active preparation derived from one or more plants. In the context of the specification by “active” it is meant that the extract is capable of producing a desired therapeutic or cosmetic benefit as disclosed herein. An extract is obtained by a process of “extraction” which will be understood by those skilled in the art as, in general terms, comprising treating plant material with a solvent, a liquid, or a supercritical fluid to dissolve the active preparation and separate the same from residual unwanted plant material. An extract may be in liquid form (for example as a decoction, solution, infusion or tincture) or solid form (for example as a powder or granules). The term “combination extract” as used herein refers to an extract prepared from more than one plant species. In a combination extract the plant material from each of the plant species may be subjected to the extraction process together or separately. That is, material from some or all of the species may be combined prior to addition of the solvent, liquid or supercritical fluid, and/or material from some or all of the species may be independently treated with a solvent, liquid or supercritical fluid and the preparations so obtained are subsequently combined. As such, the same or different solvents (or liquids or supercritical fluids) may be used to extract the active preparation from the different species. The terms “extract” and “combination extract” may be used interchangeably throughout the specification.

The terms “promoting”, “promotion” and variations thereof as used herein in the context of health and/or appearance of the skin and/or an epidermal appendage refer to the ability of a composition of the invention to enhance or increase the rate or degree of action of natural physiological process(es) involved in improving the health and/or appearance of the skin and/or an epidermal appendage.

As used herein the terms “treating”, “treatment”, “preventing” and “prevention” refer to any and all uses which remedy a condition or symptoms, prevent the establishment of a condition or disease, or otherwise prevent, hinder, retard, or reverse the progression of a condition or disease or other undesirable symptoms in any way whatsoever. Thus the terms “treating” and “preventing” and the like are to be considered in their broadest context. For example, treatment does not necessarily imply that a patient is treated until total recovery.

As used herein the terms “improve” and “improving” refer to the ability to achieve a measurable improvement in the appearance or texture of the skin or an epidermal appendage, or otherwise prevent, hinder, retard, or reverse the deterioration of the appearance of the skin or epidermal appendage in any way whatsoever. Thus the terms “improve”, “improving” and the like are to be considered in their broadest context.

As used herein the terms “effective amount” and “effective dose” include within their meaning a non-toxic but sufficient amount or dose of an agent or compound to provide the desired effect. The exact amount or dose required will vary from subject to subject depending on factors such as the species being treated, the age and general condition of the subject, the severity of the condition being treated, the particular agent being administered and the mode of administration and so forth. Thus; it is not possible to specify an exact “effective amount” or “effective dose”. However, for any given case, an appropriate “effective amount” or “effective dose” may be determined by one of ordinary skill in the art using only routine experimentation.

In the context of this specification, the term “cosmetically and pharmaceutically acceptable” means that the compound to which it refers is suitable for use in contact with tissues of the body without undue toxicity, incompatibility, instability, irritation, allergic response, and the like, commensurate with a reasonable benefit/risk ratio.

As used herein the term “subject” includes humans, primates, livestock (eg. sheep, pigs, cattle, horses, donkeys), laboratory test animals (eg. mice, rabbits, rats, guinea pigs), companion animals (eg. dogs, cats), show animals (eg. dogs, cats, pigs, cattle, sheep, horses) and captive wild animals (eg. foxes, kangaroos, deer).

The present invention is predicated on the inventors' surprising finding, as exemplified herein, that the use of compositions comprising extracts of various plants possess biological activity when administered to mammalian skin. Accordingly, disclosed herein are compositions and methods for maintaining and improving the health and/or appearance of the skin and/or epidermal appendages. For the purposes of the present invention epidermal appendages include, but are not limited to, hair, hair follicles, nails, cuticles, and teeth.

The administration of a composition of the present invention may maintain or improve the health and/or appearance of the skin and/or an epidermal appendage of a subject by providing improvements including:
anti-ageing effects
retardation and reduction of wrinkles or fine lines
improved radiance
improved skin tone
improved skin texture and firmness
reduced skin sensitivity
reduced skin roughness
protection against oxidative stress
promotion of collagen production
promotion of elastin production
skin and/or epidermal appendage lightening effects
promotion of skin repair
reduction of trans-epidermal water loss
improvement in collagen cross-linking
supporting the skin barrier function
reduction of acne scars
reduction of age spots
reduction of blotchiness, broken capillaries, intracutaneous and/or subcutaneous bleeding
reduction of visible pores
reduction in stretch marks
promotion of skin regeneration
promotion of desquamation
restoration of the hydrolipid film
promotion of lipid synthesis
promotion of skin and/or epidermal appendage healing
restoration of the physiological balance of the skin and/or epidermal appendage
promotion of cell renewal
increasing of natural moisturising factors in the skin

Accordingly, one aspect of the present invention provides a composition comprising a biologically active extract of Vigna sp., Terminalia sp., Cocos nucifera and/or Hibiscus sp. for improving the health and/or appearance of the skin and/or epidermal appendages.

The inventors have surprisingly found that extract(s) of Vigna sp., Cocos nucifera, Terminalia sp. and/or Hibiscus sp. alone or in various combination promote the body's natural process of regenerating dermal and epidermal tissue.

Accordingly, compositions comprising extracts of Vigna sp., Cocos nucifera, Terminalia sp. and/or Hibiscus sp. are useful for maintaining and improving the health and/or appearance of the skin and/or epidermal appendages. The improvement may be, but is not limited to, a reduction in the appearance of fine line and wrinkles, an increase in skin radiance, an increase in skin tone, an increase in skin texture and/or firmness, a reduction in acne scars, a decrease in age spots, a reduction in the blotchiness, broken capillaries, intracutaneous and/or subcutaneous bleeding of the skin, an increase in the lightness of skin and/or an epidermal appendage, a reduction of visible pores, a reduction in stretch marks, an increase in skin elasticity and/or a reduction in skin sagging, a reduction in skin roughness, a reduction in skin sensitivity, restoration of the physiological balance of the skin and/or an epidermal appendage, an increase in trans-epidermal water loss, or an increase in the natural moisturising factors in the skin.

The Vigna sp. extract may be derived from one or more of, but is not limited to, Vigna aconitifolia, Vigna angularis, Vigna caracalla, Vigna debilis, Vigna dinteri, Vigna lanceolata, Vigna luteola, Vigna marina (Burm.) Merr., Vigna maritime, Vigna mungo, Vigna o-wahuensis, Vigna parkeri, Vigna radiata, Vigna speciosa, Vigna subterranea, Vigna trilobata, Vigna umbellata, Vigna unguiculata or Vigna vexillata. In one embodiment the Vigna is Vigna marina (Burm.) Merr.

The Terminalia sp. extract may be derived from one or more of, but is not limited to, Terminalia acuminate, Terminalia alata, Terminalia altissima, Terminalia amazonia, Terminalia angustifolia, Terminalia arborea, Terminalia arbuscula, Terminalia archipelagi, Terminalia arjuna, Terminalia australis, Terminalia avicennioides, Terminalia bellirica, Terminalia bialata, Terminalia brachystemma, Terminalia brassii, Terminalia bucidoides, Terminalia buceras (Bucida buceras), Terminalia bursarina, Terminalia calamansanai, Terminalia catappa, Terminalia chebula, Terminalia cherrieri, Terminalia ciliate, Terminalia citrina, Terminalia copelandii, Terminalia corticosa, Terminalia eddowesii, Terminalia edulis, Terminalia elliptica, Terminalia eriostachya, Terminalia erythrophylla, Terminalia ferdinandiana, Terminalia foetidissima, Terminalia franchetii, Terminalia glabrescens, Terminalia glaucifolia, Terminalia hararensis, Terminalia hecistocarpa, Terminalia intermedia, Terminalia ivorensis, Terminalia januariensis, Terminalia kaembachii, Terminalia kangeanensis, Terminalia kuhlmannii, Terminalia latifolia, Terminalia mantaly, Terminalia molinetii Terminalia muelleri, Terminalia myriocarpa, Terminalia nitens, Terminalia novocaledonica, Terminalia oblongata, Terminalia obovata, Terminalia oliveri, Terminalia paniculata, Terminalia parviflora, Terminalia pellucida, Terminalia phanerophlebia, Terminalia phellocarpa, Terminalia prunloides, Terminalia reitzii, Terminalia rerei, Terminalia schimperiana, Terminalia sericea, Terminalia seriocarpa, Terminalia subspathulata, Terminalia superba, Terminalia tripteroides or Terminalia volucris. In one embodiment the Terminalia is Terminalia catappa L.

The Hibiscus sp. extract may be derived from one or more of, but is not limited to, Hibiscus acetosella, Hibiscus brackenridgei, Hibiscus cannabinus, Hibiscus diversifolius, Hibiscus lavaterioide, Hibiscus ludwigii, Hibiscus macrophyllus, Hibiscus macropodus, Hibiscus elates, Hibiscus escobariae, Hibiscus ficulneus, Hibiscus paramutabilis, Hibiscus pedunculatus, Hibiscus platanifolius, Hibiscus radiatus, Hibiscus rosa-sinensis, Hibiscus sabdariffa, Hibiscus schizopetalus, Hibiscus scottii, Hibiscus socotranus, Hibiscus sinosyriacus, Hibiscus splendens, Hibiscus stenanthus, Hibiscus striatus, Hibiscus syriacus, Hibiscus tiliaceus, Hibiscus trilobus, or Hibiscus waimeae. In one embodiment the Hibiscus is Hibiscus tiliaceus L.

Compositions of the invention comprise extracts that may be aqueous, oil and/or organic solvent based extracts, obtained by single, combined and/or successive extraction of any available plant material such as leaves, roots, bark, stems, fruits, shoots, nuts, husks of nuts, seeds, seed capsules, flowers, vine and/or wood. Suitable extraction processes, and suitable solvents and liquids for extraction are known to those skilled in the art. Aqueous solvents (for example water, acids, bases); lipids (for example, synthetic lipids or lipids derived from nature or synthetic copies thereof) and oils (for example Cocos nucifera); and organic solvents, which can be polar (such as alcohols for example ethanol), non-polar (for example hexane) and/or halogenated (for example dichloromethane), used for extraction can either be used sequentially for extraction or in combination mixture. Importantly, as exemplified herein, the activity of the extract is maintained when extracted into Cocos nucifera oil, polar solvents (e.g. ethanol) or non-polar solvents (e.g. isopentane). Supercritical fluid extraction using, for example, supercritical nitrogen or carbon dioxide, may also be used in accordance with the invention to obtain extracts.

Further, it will be appreciated by those skilled in the art that an extract of the invention may be subjected to one or more post extraction steps to, for example, increase or maintain the stability of the extract, modify or change the physical form of the extract or assist in formulating the extract into a composition for administration to a subject. By way of example only a liquid form extract may be lyophilised to produce a solid form of the extract.

Extracts of the present invention may be derived from any suitable plant material. Suitable plant material includes leaves, roots, bark, stems, fruits, shoots, nuts, husks of nuts, seeds, seed capsules, flowers, vine or wood. The plant material may be, for example, fresh, dried or freeze dried. For any given plant species more than one plant material may be used for the production of extracts. Where derived from Vigna marina (Burm.) Merr., Terminalia catappa L., and Hibiscus tiliaceus L., typically the extract is a leaf, vine and/or bean extract. Where derived from Cocos nucifera typically the extract is a fresh husk and/or nut extract.

Compositions comprising extract(s) of the invention may be administered in accordance with the present invention in the form of cosmetic or pharmaceutical compositions, which compositions may comprise one or more cosmetically or pharmaceutically acceptable carriers, excipients or diluents. Compositions may further comprise other therapeutic or cosmetic agents for example, but not limited to, antibiotics, antimicrobial agents, antiseptics, anaesthetics, moisturisers or cosmetic bases. Such compositions may be administered in any convenient or suitable route such as by parenteral, oral, nasal or topical routes. Typically for the purposes of achieving the therapeutic and cosmetic benefits as disclosed herein, the route of administration may be topical. Alternatively, administration by injection, for example subcutaneous injection, may also be appropriate depending on the desired outcome.

It will be understood that the specific dose level of a composition of the invention for any particular individual will depend upon a variety of factors including, for example, the activity of the specific extract(s) employed, the age, body weight, general health and diet of the individual to be treated, the time of administration, the stability of the composition and extract(s), the site of application on the body, and combination with any other treatment or therapy. Single or multiple administrations can be carried out with dose levels and pattern being determined as required depending on the circumstances and the individual to be treated. Suitable dosage regimes can readily be determined by the skilled addressee. A broad range of doses may be applicable. Considering a human subject, for example, from about 0.01 mg to about 100 mg of extract may be administered per kilogram of body weight per day. Dosage regimens may be adjusted to provide the optimum therapeutic response. For example, several divided doses may be administered hourly, daily, weekly, monthly or at other suitable time intervals or the dose may be proportionally reduced as indicated by the exigencies of the situation.

Generally, an effective dosage is expected to be in the range of about 0.0001 mg to about 1000 mg per kg body weight per 24 hours; typically, about 0.001 mg to about 750 mg per kg body weight per 24 hours; about 0.01 mg to about 500 mg per kg body weight per 24 hours; about 0.1 mg to about 500 mg per kg body weight per 24 hours; about 0.1 mg to about 250 mg per kg body weight per 24 hours; about 1.0 mg to about 250 mg per kg body weight per. 24 hours. More typically, an effective dose range is expected to be in the range about 1.0 mg to about 200 mg per kg body weight per 24 hours; about 1.0 mg to about 100 mg per kg body weight per 24 hours; about 1.0 mg to about 50 mg per kg body weight per 24 hours; about 1.0 mg to about 25 mg per kg body weight per 24 hours; about 5.0 mg to about 50 mg per kg body weight per 24 hours; about 5.0 mg to about 20 mg per kg body weight per 24 hours; about 5.0 mg to about 15 mg per kg body weight per 24 hours.

Topical formulations typically comprise one or more compositions of the invention together with one or more acceptable carriers, and optionally any other therapeutic ingredients. The compositions of the invention are incorporated into topical vehicles, such as cosmetic vehicles, for application to the skin and/or an epidermal appendage of a subject.

The extract of Vigna sp., Cocos nucifera, Terminalia sp. and/or Hibiscus sp. may be present in an amount between about 0.001% (w/w) and about 15% (w/w), or between about 0.001% and about 12%, or between about 0.001% and about 10%, or between about 0.005% and about 10%, or between about 0.01% and about 10%, or between about 0.05% and about 10%, or between about 0.05% and about 5% in the topical vehicle.

Suitable cosmetic vehicles include, but are not limited to lotions, creams, gels, ointments or pastes for example: make up, products intended for application to the lips, face masks and scrubs, hair gels and mousses, hair tints, dyes and bleaches, products for waving, straightening and fixing hair, cleansing products such as lotions, skin softeners, powders and shampoos, conditioning products such as lotions, creams, oils, anti-ageing products, skin whitening products, hairdressing products such as lotions and lacquers, deodorants, antiperspirants, cleansers such as toilet soap, deodorant soap, astringent and skin washes, shaving products such as creams, foams and lotions, bath and shower preparations such as salts, foams, oils, gels, etc., depilatories, after-bath powders, hygienic powders, moisturising products such as creams, lotions, gels and foams, sunscreens and sunbathing products, decorative cosmetics, such as lipsticks, eyeshadows, mascaras, eyeliner, nail lacquer, nailcare products, anti-wrinkle products and anti-ageing products. The cosmetic product may be a leave-on composition, a rinsing composition or a cleansing composition.

Examples of cosmetically or pharmaceutically acceptable carriers or diluents are demineralised or distilled water; saline solution; vegetable based or derived oils and lipids such as peanut oil, almond oil, safflower oil, olive oil, cottonseed oil, maize oil, sesame oil, arachis oil or coconut oil; silicone oils, including polysiloxanes, such as methyl polysiloxane, phenyl polysiloxane and methylphenyl polysiloxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrridone; agar; carrageenan; gum tragacanth or gum acacia, and petroleum jelly. Typically, the carrier or carriers will form from 10% to 99.9% by weight of the compositions.

Lotions according to the present invention include those suitable for application to the skin or to an epidermal appendage. Lotions or liniments for application to the skin may also include an agent to hasten drying and to cool the skin, such as an alcohol or acetone, and/or a moisturiser such as glycerol, or oil such as coconut oil, castor oil or arachis oil.

Creams, ointments or pastes according to the present invention are semi-solid formulations of the extract for external application. They may be made by mixing the extract in finely-divided or powdered form, alone or in solution or suspension in an aqueous or non-aqueous fluid, with a greasy or non-greasy basis. The basis may comprise hydrocarbons such as hard, soft or liquid paraffin, glycerol, beeswax, a metallic soap; a mucilage; an oil of natural origin such as coconut, almond, corn, arachis, castor or olive oil; wool fat or its derivatives, or a fatty acid such as stearic or oleic acid together with an alcohol such as propylene glycol or macrogols.

The compositions may further comprise one or more UV filter and/or sunblocking agents so as to provide a sun protection factor (SPF) for the blocking of harmful UVB rays, a UVA protection factor for the blocking of harmful and skin-aging effects of ultraviolet light. Suitable UV filter and sunblocking agents include, but are not limited to, organic and inorganic UV filter and sunblocking agents, for example zinc oxide, titanium dioxide, ethyl methoxycinnamate, dimethoxycinnamate, glycol salicylate, butyl PABA, cinoxate and Heliogard 335. The UV filter and/or sunblocking agent may be a herbal component, for example but not limited to a wild pansy extract which acts a UV filter. Additional UV filter and/or sunblocking agents include those disclosed in, for example, “Cosmetic, Science and Technology” 2nd Edition (1972), Vol. 1, Chapter VIII, pages 189 et seq or in, for example, the current edition of the “International Cosmetic Ingredient Dictionary and Handbook”, published and distributed by the Personal Care Products Council. Exact amounts of the UV filter or sunblocking agents will vary depending upon the sunscreen chosen and the desired SPF and UVA protection factor.

The compositions of the invention may be included in topical vehicles in an amount between about 0.001% (w/w) and about 90% (w/w), or between about 1% (w/w) and about 50% (w/w), or between about 1% (w/w) and about 40% (w/w), or between about 1% (w/w) and about 20% (w/w) or about 0.01%, 0.5%, 1%, 2%, 3%, 4%, 5%, 6%, 7%, 8%, 9%, 10%, 11%, 12%, 13% 14%, 15%, 16%, 17%, 18%, 19%, 20%, 25%, 30%, 35%, 40%, 45%, 50%, 55%, 60%, 65%, 70%, 75%, 80%, 85%, 90%.

The compositions may further comprise additional antioxidants, moisturisers, essential oils, additional plant extracts, skin conditioning agents, herbal extracts, humectants, surfactants, vitamins and micronutrients, thickening agents, preservatives, exfoliants, anti-inflammatories, fragrances, and the like.

Examples of antioxidants include, but are not limited to, water-soluble antioxidants such as sulfhydryl compounds and their derivatives (for example sodium metabisulfite and N-acetyl-cysteine), lipoic acid and dihydrolipoic acid, resveratrol and lactoferrin. Oil-soluble antioxidants include, but are not limited to butylated hydroxytoluene, retinoids, tocopherols and ubiquinone.

Examples of moisturisers include but are not limited to: fatty acids, lanolin, cetyl palmitate, castor oil, jojoba seed oil, grape seed oil, sunflower seed oil, safflower seed oil, diglycerin, oleic acid, dimethicone copolyol, dextrin, jojoba esters, panthenol, squalene, coconut oil, gelatin, cocoa butter, hydrogenated lecithin, isopropyl isostearate, hydrogenated vegetable oil, glycerol polymers, glycerin, sorbitan palmitate, petrolatum, glyceryl distearate and ceramides.

Essential oils may enhance the emollient and penetration properties of the composition. Essential oils include, but are not limited to lemongrass oil, tea tree oil, thyme oil, lavender oil and alpha Bisabolol. A person skilled in the art will readily appreciate the essential oils may be used to enhance the emollient and penetration properties of the composition.

Additional plant extracts include, but are not limited to Horse chestnut extract, Bamboo extract and squalene. The skilled addressee will readily appreciate that further plant extracts may be used to confer one or more desirable property to the composition.

When present in the composition, the additional plant extracts and/or herbal extracts may be present in an amount between about 0.005% (w/w) and about 20% (w/w), or between about 0.05% and about 15%, or between about 0.05% and about 10%.

Humectants include but are not limited to glycerol, sorbitol, polyethylene glycol, and mono- and oligomeric sugars.

Vitamins and micronutrients include but are not limited to vitamin A, pro vitamin A, vitamin B₁, vitamin B₂, vitamin B₃, vitamin B₄, vitamin B₅, vitamin B₆, vitamin B₁₂, vitamin D, vitamin D₂, vitamin D3, tocopherol (vitamin E), vitamin F, vitamin K₁; macrominerals including but not limited to calcium, chloride, magnesium, phosphorus, sodium and iron; and trace minerals including but not limited to boron, cobalt, chloride, chromium, copper, fluoride, iodine, iron, manganese, molybdenum, selenium and zinc.

Thickening agents include but are not limited to carbomers, hydroxymethyl cellulose, xanthan gum, guar gum, chitosan and polyquaternium-10.

Preservatives include but are not limited to totarol, parabens, quaternary ammonium chlorides, benzalkonium chlorides, isothiazolinones, parabens, chloroxylenol; chlorhexidine, phenoxyethanol, benzyl alcohol, phenethyl alcohol, benzoic add and salts thereof, chlorobutanol, sorbic acid and salts thereof, triclosan and triclocarban.

Skin conditioning agents include for example, hydroxyacids. The term “hydroxyacids” includes both α- and β-hydroxyacids. Suitable hydroxyacids include, but are not limited to α-hydroxyacids such as lactic acid, glycolic acid, citric acid, α-hydroxyoctanoic acid, tartaric acid, glucoronic acid, α-hydroxybutyric acid, malic acid, mandelic acid and pyruvic acid. Suitable β-hydroxy acids include but are not limited to salicylic acid, β-hydroxy butyric acid and carnitine.

The compositions also may be impregnated into transdermal patches, plasters, and dressings such as bandages or hydrocolloid dressings, preferably in liquid or semi-liquid form.

In particular circumstances, for example in maintaining and improving the health of skin, administration of compositions by injection, typically subcutaneous injection may be appropriate. Pharmaceutical forms suitable for injectable use include sterile aqueous solutions (where water soluble) or dispersions and sterile powders for the extemporaneous preparation of sterile injectable solutions. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The preventions of the action of microorganisms can be brought about by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filter sterilisation. Generally, dispersions are prepared by incorporating the various sterilised extract(s) into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the extract plus any additional desired extract from previously sterile-filtered solution thereof.

The present invention contemplates combination therapies, wherein compositions as described herein are coadministered with other suitable agents or treatments which may facilitate the desired improvement. For example, one may seek to aid the maintenance and improvement of the health and/or appearance of skin and/or epidermal appendages with antibiotics, antimicrobial agents, or other skin healing agents in combination with compositions of the invention. By “coadministered” is meant simultaneous administration in the same formulation or in two different formulations via the same or different routes or sequential administration by the same or different routes. By “sequential” administration is meant a time difference of from seconds, minutes, hours or days between the administration of the two types of agents. The agents may be administered in any order. For example, embodiments of the present invention contemplate the administration of a lipid or an oil, for example Cocos nucifera oil, to the skin or epidermal appendage prior to, or after, the composition is applied.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The present invention will now be described with reference to the following specific examples, which should not be construed as in any way limiting the scope of the invention.

### EXAMPLES

### Example 1

### Preparation of a Combination Extract of Vigna marina (Burm.) Merr., Cocos nucifera L., Terminalia catappa L. and Hibiscus tiliaceus L. in coconut oil Coconut oil (200 mL) was added to fresh shredded leaves of Vigna marina (Burm.) Merr. (100 g) and fresh shredded leaves of Terminalia catappa L. (100 g). The mixture was placed in a crucible in a water bath at 100° C. for 20 min. The mixture was removed from the heat and immediately filtered and pressed to extract the coconut oil. The resultant coconut oil, containing extracts of Vigna marina (Burm.) Merr. and Terminalia catappa L., was added to the fresh crushed husk of a green nut of Cocos nucifera L. (100 g) and the shaved bark of Hibiscus tiliaceus L. (100 g). The mixture was left to settle for 4 h and then filtered and pressed to extract the coconut oil.

The coconut oil was then inverted and stored at less than 20° C. until the coconut oil solidified. Any remaining moisture or solid in the mixture were removed by decanting from the solidified coconut oil.

The coconut oil was then heated in a hot water bath at approximately 56° C. and filtered. The resultant filtrate contained a combination extract of Vigna marina (Burm.) Merr., Cocos nucifera L., Terminalia catappa L. and Hibiscus tiliaceus L.

### Example 2

### Treatment of Oestrogen Deficient Skin

A study was carried out to assess the effect of an extract of Vigna marina (Burm.) Merr., Cocos nucifera L., Terminalia catappa L. and Hibiscus tiliaceus L. prepared as described in Example 1, on estrogen deficient skin. In the study a daily dose 1 mL of the extract was applied topically to the epithelial surface of the backs of 18 month old, oestrogen deficient (ovaries removed at age 6 weeks) rats (10 animals). The extract was applied daily for a total of 7 days. The control animals were 18 month old, oestrogen deficient (ovaries removed at age 6 weeks) rats with no topical application of the extract (10 animals). At 7 days the histology of the treated and control skin was assessed and the epithelial thickness determined.

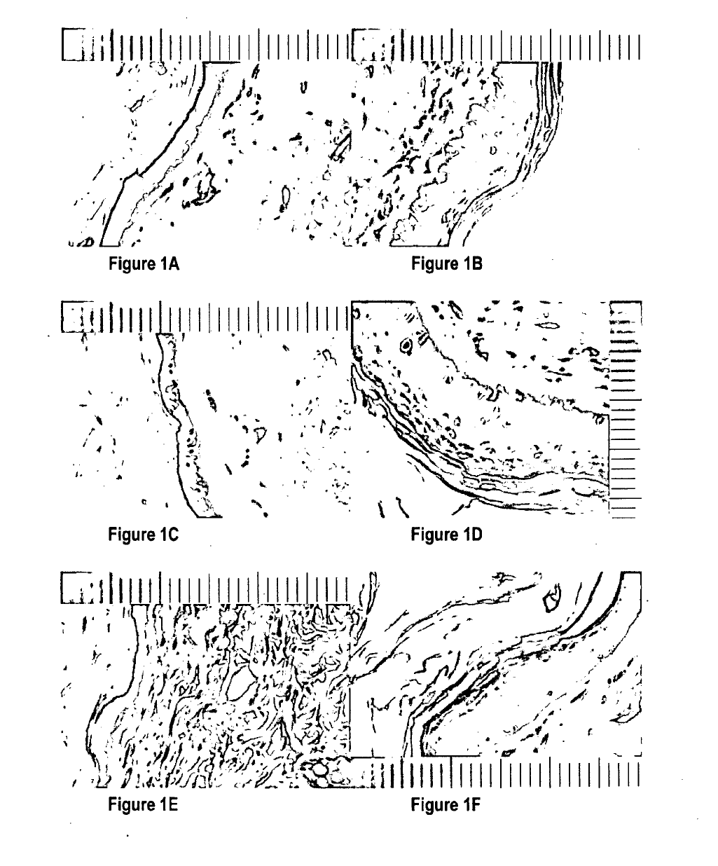

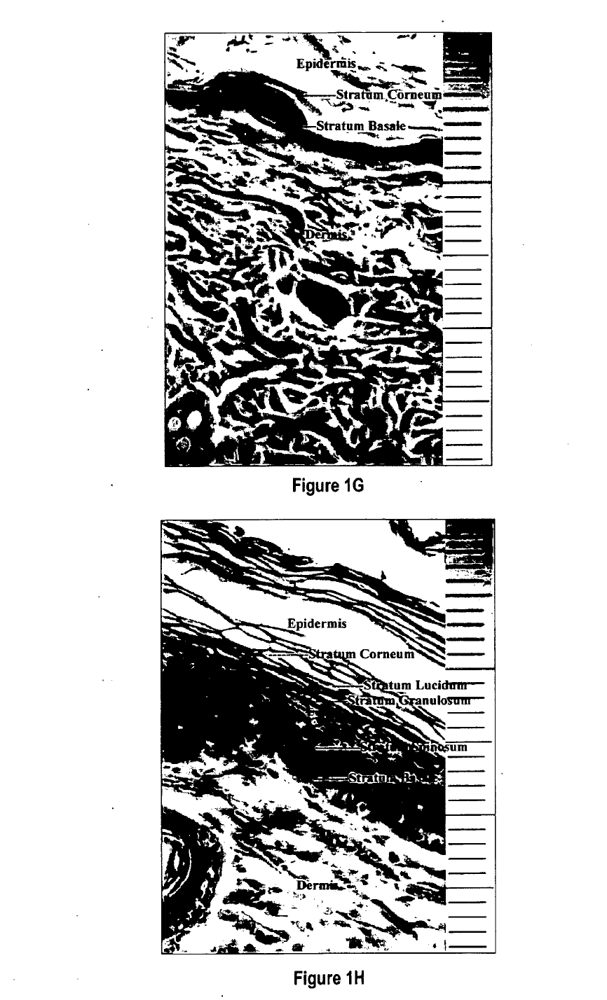

The histological profile of treated and control skin from individual animals at 7 days is shown in FIGS. 1A to 1H. The histology of the treated skin compared to the control shows a thick highly cellular epithelium and a relative increase in cellularity of the collagen matrix.

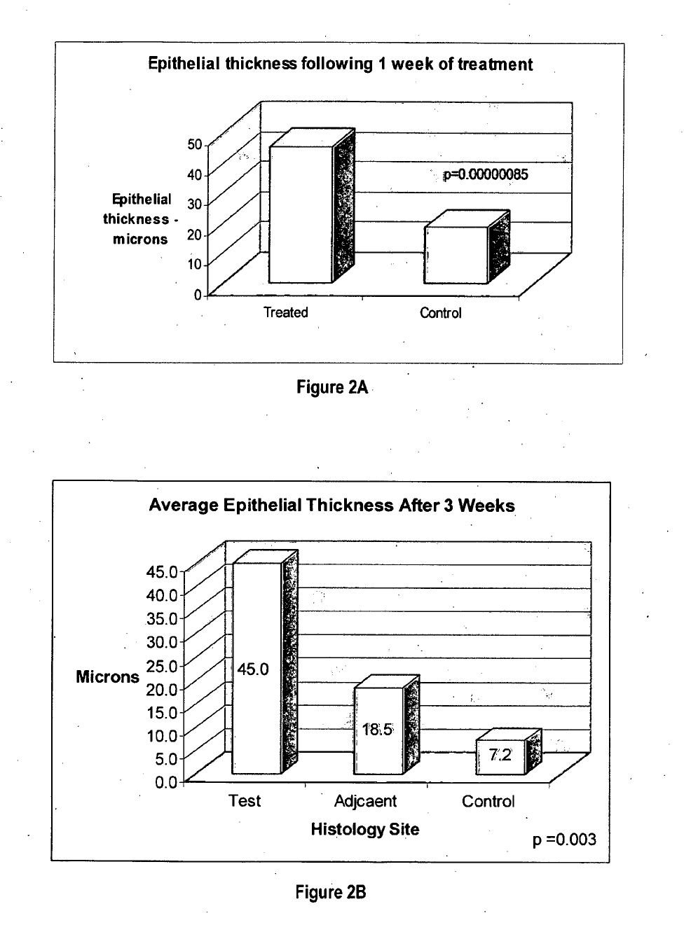

A graphical representation of the epithelial thickness of the skin of treated and untreated animal is shown in FIG. 2A. An average increase of 145% in the epithelial thickness of the skin in treated compared to untreated animals was observed.

An identical study was carried out with the exception that the extract was applied daily for 3 weeks (10 animals). At 21 days the epithelial thickness of the treated skin (site of application and adjacent site) and untreated (control) skin was determined. This study demonstrates that the increase in epithelial thickness and cellularity of the collagen matrix observed over 7 days (see above) are maintained over 21 days of treatment. Epithelial thickness following 21 days of treatment is shown in FIG. 2B. The results in FIG. 2B show that the effect is not only seen in the area of application but also in the areas adjacent to the applied area implying that only a small dose is required to bring about the effects to aged oestrogen deficient skin.

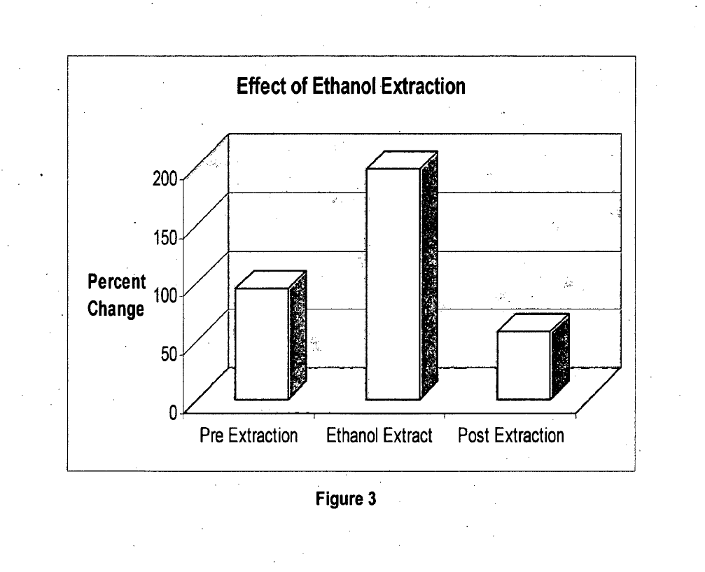

It was also demonstrated that the biological activity of the coconut oil based extract is soluble in ethanol. The extract in coconut oil (pre-extraction) was vigorously mixed 1:1 with ethanol for 5 min until the mixture was homogeneous and then left to stand for 5 min, the mixture was then frozen and the ethanol removed (ethanol extract) to leave coconut oil (post extraction) only. At 7 days the epithelial thickness was determined. FIG. 3 shows the percentage change in epithelial thickness of treated oestrogen deficient skin following the treatment. The results demonstrate that the activity of the extract is maintained when extracted into ethanol.

### Example 3

### Preparation of Extracts of Vigna marina (Burm.) Merr.

3.1: Vigna marina (Burm.) Merr. Extract in Coconut Oil

The fresh crushed leaves, vine and bean of Vigna marina (Burm.) Merr. (100 g) were immersed in coconut oil (100 mL). The extract was prepared by cold pressing the Vigna marina (Burm.) Merr. and coconut oil mixture; or by heating the mixture. The cold pressing was carried out by placing the Vigna marina (Burm.) Merr. and coconut oil mixture inside a new coffee plunger and leaving the mixture to settle for 1 h, the plunger was then depressed thereby extracting the oil. The mixture was left to settle for a further 5 min and the plunger depressed again to release more oil. This process was repeated 5 times until no further oil was obtained. When the extract was prepared by heating, the Vigna marina (Burm.) Merr. and coconut oil mixture was heated in a crucible in a water bath at 100° C. for 20 min, removed from the heat, and then immediately filtered and pressed to extract the coconut oil. Any remaining moisture or solid in the extract after cold pressing or heating and filtering were removed by inverting the coconut oil extract and storing it at less than 20° C. until the coconut oil solidified and then decanting the solidified coconut oil; or by decanting in a separating funnel.

3.2: Vigna marina (Burm.) Merr. Extract in Coconut Oil Following Extraction in Ethanol

The fresh crushed leaves, vine and bean of Vigna marina (Burm.) Merr. (1000 g) were immersed in 95-100% ethanol (2000 mL) and left to steep for 24 h. The ethanol extraction of Vigna marina (Burm.) Merr. was agitated with coconut oil (250 mL). The coconut oil was decanted from the solution following standing for at least 2 h in a decanting vessel; or by the dilution of the ethanol solution from 95% to a lower concentration, nominally but not necessarily 50% ethanol and then decanted after settling of the layers. Any remaining moisture or solid in the mixture were removed by inverting the coconut oil extract and storing it at less than 20° C. until the coconut oil solidified and then decanting the solidified coconut oil; or by decanting in a separating funnel.

3.3 Vigna marina (Burm.) Merr. Extract in Coconut Oil Following Extraction in Ethanol and Isopentane

The fresh crushed leaves, vine and bean of Vigna marina (Burm.) Merr. (1000 g) were immersed in 95-100% ethanol (200.0 mL) and left to steep for 0.6 h, the ethanol was then removed and the Vigna marina (Burm.) Merr. steeped in a second aliquot of ethanol (2000 mL) for a further 6 h.

The resultant ethanol was agitated with hexane, pentane, methyl butane or an equivalent hydrocarbon or fluoro/chloro/bromo-hydrocarbon (250 mL). The hydrocarbon was decanted from the solution following standing for at least 2 h in a decanting vessel; or by the dilution of the ethanol solution from 95% to a lower concentration, nominally but not necessarily 50% ethanol and then decanted after settling of the layers.

Coconut oil (250 mL) was added to the hydrocarbon solution and heated above the boiling point of the hydrocarbon (for example when hexane was used the solution was heated to 67° C.) to evaporate the hydrocarbon. 95% Ethanol (250 mL) was added to the hydrocarbon solution and heated above the boiling point of the hydrocarbon.

3.4: Vigna marina (Burm.) Merr. Extract in Hydrocarbon Following Extraction in Ethanol

The fresh crushed leaves, vine and bean of Vigna marina (Burm.) Merr. (1000 g) were immersed in 95-100% ethanol (2000 mL) and left to steep for 6 h, the ethanol was then removed and the Vigna marina (Burm.) Merr. steeped in a second aliquot of ethanol (2000 mL) for a further 6 h. The resultant ethanol solution was agitated with hexane, pentane, methyl butane or an equivalent hydrocarbon or fluoro/chloro/bromo-hydrocarbon (250 mL). The hydrocarbon was decanted from the solution following standing for at least 2 h in a decanting vessel; or by the dilution of the ethanol solution from 95% to a lower concentration, nominally but not necessarily 50% ethanol and then decanted after settling of the layers.

3.5: Viva marina (Burm.) Merr. Extract in Ethanol Following Extraction in Ethanol and Hydrocarbon

The fresh crushed leaves, vine and bean of Vigna marina (Burm.) Merr. (1000 g) were immersed in 95-100% ethanol (2000 mL) and left to steep for 6 h, the ethanol was then removed and the Vigna marina (Burm.) Merr. steeped in a second aliquot of ethanol (2000 mL) for a further 6 h. The resultant ethanol solution was agitated with hexane, pentane, methyl butane or an equivalent hydrocarbon or fluoro/chloro/bromo-hydrocarbon (250 mL). The hydrocarbon was decanted from the solution following standing for at least 2 h. in a decanting vessel; or by the dilution of the ethanol solution from 95% to a lower concentration, nominally but not necessarily 50% ethanol and then decanted after settling of the layers.

95% Ethanol (250 mL) was added to the hydrocarbon solution and heated above the boiling point of the hydrocarbon (for example when hexane was used the solution was heated to 67° C.) to evaporate the hydrocarbon.

### Example 4

### Preparation of Extracts of Cocos nucifera L.

4.1: Cocos nucifera L. Extract in Coconut Oil

The extract was prepared as described for Vigna marina (Burm.) in Example 3.1 with the exception that the fresh husk of the young nut of Cocos nucifera L. (100 g) was used.

4.2: Cocos nucifera L. Extract in Coconut Oil Following Extraction in Ethanol

The extract was prepared as described for Vigna marina (Burm.) in Example 3.2 with the exception that the fresh husk of the young nut of Cocos nucifera L. (1000 g) was used.

4.3: Cocos nucifera L. Extract in Coconut Oil Following Extraction in Ethanol and Hydrocarbon

The extract was prepared as described for Vigna marina (Burm.) in Example 3.3 with the exception that the fresh husk of the young nut of Cocos nucifera L. (1000 g) was used.

4.4: Cocos nucifera L. Extract in Hydrocarbon Following Extraction in Ethanol

The extract was prepared as described for Vigna marina (Burm.) in Example 3.4 with the exception that the fresh husk of the young nut of Cocos nucifera L. (1000 g) was used.

4.5: Cocos nucifera L. Extract in Ethanol Following Extraction in Ethanol and Hydrocarbon

The extract was prepared as described for Vigna marina (Burm.) in Example 3.5 with the exception that the fresh husk of the young nut of Cocos nucifera L. (100 g) was used.

### Example 5

### Preparation of Extracts of Terminalia Catappa L.

5.1: Terminalia catappa L. Extract in Coconut Oil

The extract was prepared as described for Vigna marina (Burm.) in Example 3.1 with the exception that the fresh crushed leaves, vine and bean of Terminalia catappa L. (100 g) were used.

5.2: Terminalia catappa L. Extract in Coconut Oil Following Extraction in Ethanol

The extract was prepared as described for Vigna marina (Burm.) in Example 3.2 with the exception that the fresh crushed leaves, vine and bean of Terminalia catappa L. (1000 g) were used.

5.3: Terminalia catappa L. Extract in Coconut Oil Following Extraction in Ethanol and Hydrocarbon The extract was prepared as described for Vigna marina (Burm.) in Example 3.3 with the exception that the fresh crushed leaves, vine and bean of Terminalia catappa L. (1000 g) were used.
5.4: Terminalia catappa L. Extract in Hydrocarbon Following Extraction in Ethanol

The extract was prepared as described for Vigna marina (Burm.) in Example 3.4 with the exception that the fresh crushed leaves, vine and bean of Terminalia catappa L. (1000 g) were used.

5.5: Terminalia catappa L. Extract in Ethanol Following Extraction in Ethanol and Hydrocarbon

The extract was prepared as described for Vigna marina (Burm.) in Example 3.5 with the exception that the fresh crushed leaves, vine and bean of Terminalia catappa L. (1000 g) were used.

### Example 6

### Preparation of Extract of Hibiscus tiliaceus L.

6.1: Hibiscus tiliaceus L. Extract in Coconut Oil

The extract was prepared as described for Vigna marina (Burm.) in Example 3.1 with the exception that the fresh crushed leaves, vine and bean of Hibiscus tiliaceus L. (100 g) were used.

### Example 7

### Effect of Individual Extracts of Vigna marina (Burm.) Merr. and Terminalia catappa L. on the Epithelial Thickness of Skin

An assessment of the effect of each of the extracts (prepared as described in Examples 3 to 6) on the epithelial thickness of skin was carried out. A daily dose 1 mL of the extract was topically applied to the epithelial surface of the backs of 10 to 14 week old female rats for 7 days. A first control was treated with a daily dose 1 mL of coconut oil. A second control was the untreated surface of the backs of 10-14 week old female rats. At 7 days the histology of the treated and control skin was assessed.

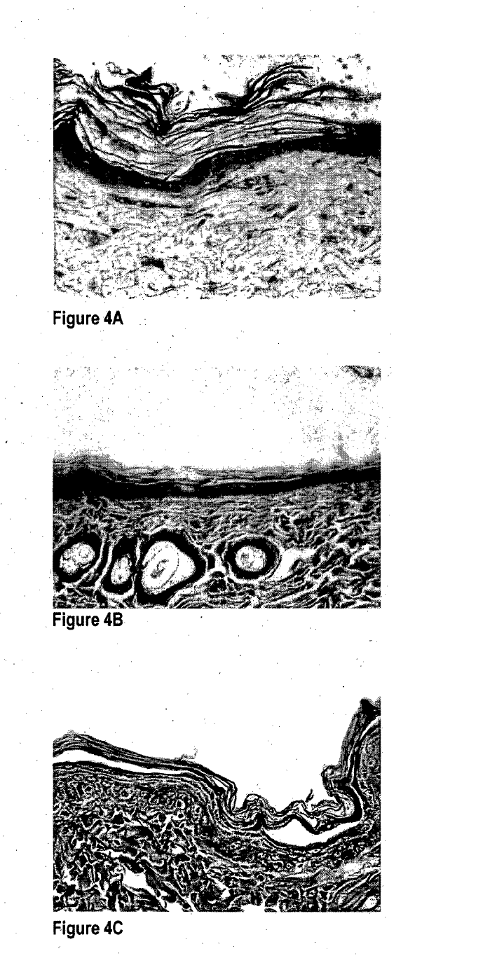

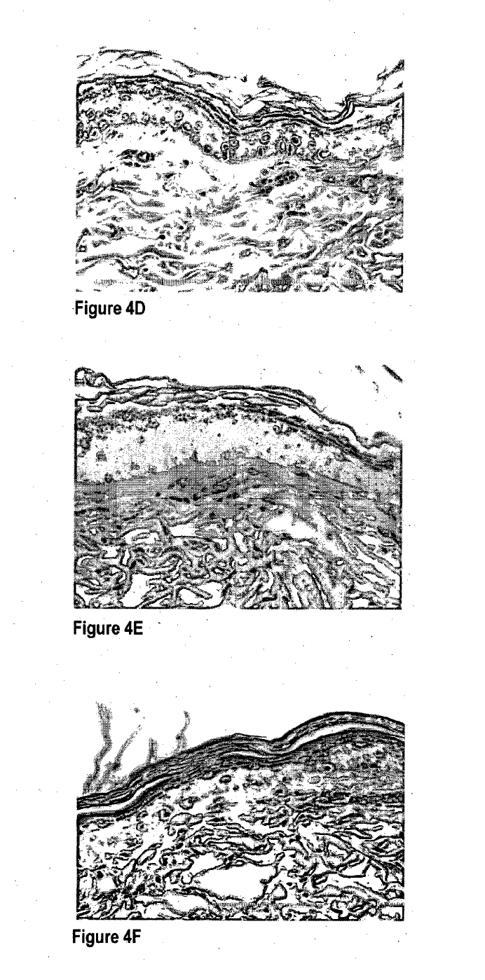

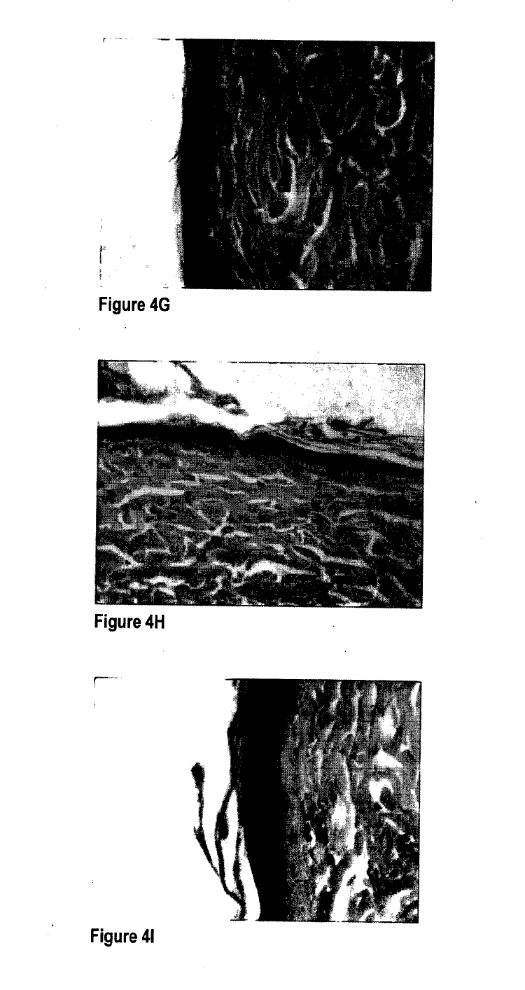

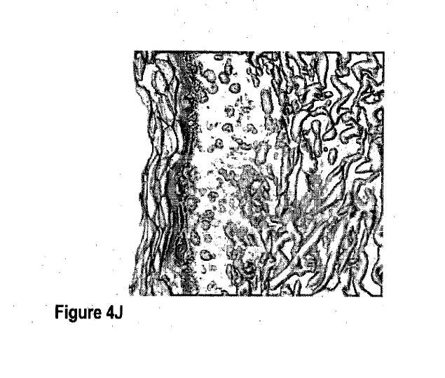

FIGS. 4A to 4J show the histology of the treated and control skin. The results show that coconut oil alone (FIG. 4B) has no obvious effect on the histological profile of the skin. The histology of skin treated with coconut oil alone is comparable to untreated skin (FIG. 4A).

FIGS. 4C to 4E show the histology after a daily application of an extract of Vigna marina (Burm.) Merr. in coconut oil (FIG. 4C); in ethanol (FIG. 4D); and in coconut oil following extraction with ethanol and hydrocarbon (FIG. 4E). The results show a marked change in epithelial cell thickness. Furthermore, there is evidence of vascular structures in the dermis and no inflammatory infiltrate. In summary, the effect of Vigna marina (Burm.) Merr. on the skin was observed in all extraction solutions, indicating the ability to concentrate the extract in alcohol, and hydrocarbon extractions.

FIGS. 4F to 4J show the histology after a daily application of an extract preparation of Terminalia catappa L. in coconut oil (FIG. 4F); in hydrocarbon (FIG. 4G); in ethanol (FIG. 4H); coconut oil following extraction with ethanol (FIG. 4I); and coconut oil following extraction with ethanol and hydrocarbon (FIG. 4J). The histological profiles show that the extracts have limited activity in the ethanol or hydrocarbon extract alone, but that activity is maintained if Terminalia catappa L. is extracted directly into coconut oil, or into ethanol or hydrocarbon and then extracted into coconut oil.

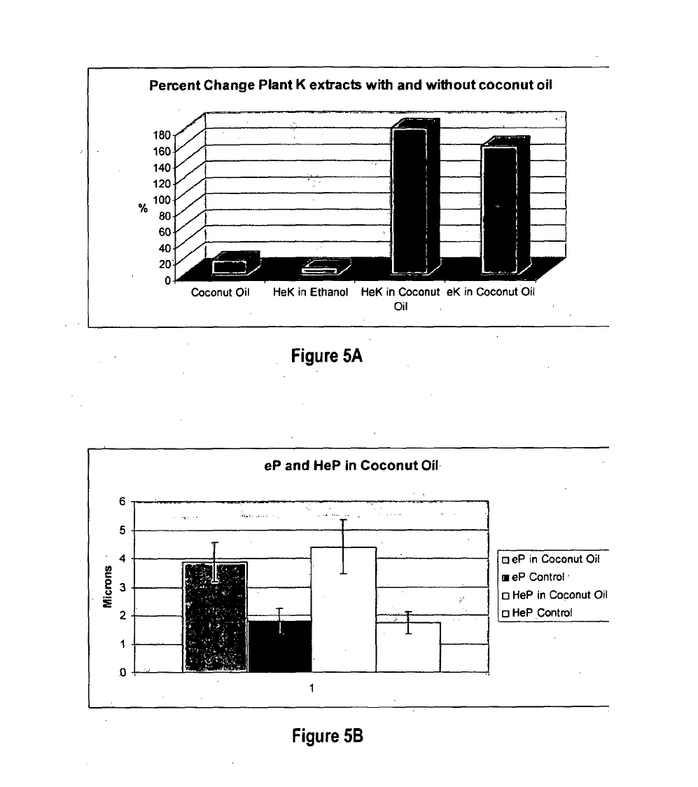

FIG. 5A shows the percentage change in epithelial thickness of skin of 10 to 14 weeks old rats following daily application for 7 days of coconut oil (Coconut Oil), Terminalia catappa L. in ethanol following extraction with ethanol and hydrocarbon (HeK in Ethanol), Terminalia catappa L. in coconut oil following extraction with ethanol and hydrocarbon (HeK in Coconut Oil), and Terminalia catappa L. in coconut oil following extraction with ethanol (eK in Coconut Oil) and FIG. 5B shows the epithelial thickness in microns of skin of 10 to 14 weeks old rats following daily application for 7 days of Vigna marina (Burm.) Merr. in coconut oil following extraction with ethanol (eP in Coconut Oil), Vigna marina (Burm.) Merr. in ethanol following extraction with ethanol (eP Control), Vigna marina (Burm.) Merr. in coconut oil following extraction with ethanol and hydrocarbon (HeP in Coconut Oil), and Vigna marina (Burm.) Merr. in ethanol following extraction with ethanol and hydrocarbon (HeP Control. FIGS. 5A and 5B indicate that the extracts have a limited effect on the epithelial thickness of skin in the ethanol or hydrocarbon extract alone, but that activity is maintained if Terminalia catappa L. or Vigna marina (Burm.) Merr. is extracted directly into coconut oil, or into ethanol or hydrocarbon and then extracted into coconut oil.

### Example 8

### Effect of Individual and Combination Extracts of Vigna marina (Burm.) Merr., Cocos nucifera L., Terminalia catappa L. and Hibiscus tiliaceus L. on the Epithelial Thickness of Skin

An assessment of the activity of each of the extracts prepared as described in Examples 3.1, 4.1, 5.1 and 6.1 was carried out. A daily dose of 1 mL of Vigna marina (Burm.) Merr. extract in coconut oil (Example 3.1); Cocos nucifera L. extract in coconut oil (Example 4.1); 1 mL of Terminalia catappa L. extract in coconut oil (Example 5.1); and/or 1 mL of Hibiscus tiliaceus L extract in coconut oil (Example 6.1) was administered topically. Each extract was applied alone or in combination with a second extract, for example 0.5 mL Vigna marina (Burm.) Merr. extract in coconut oil +0.5 mL Cocos nucifera L. extract in coconut oil to give a dose of 1 mL. The extracts were applied topically to the epithelial surface of the backs of 10-14 week old female rats. At 7 days the epithelial thickness of the skin was determined.

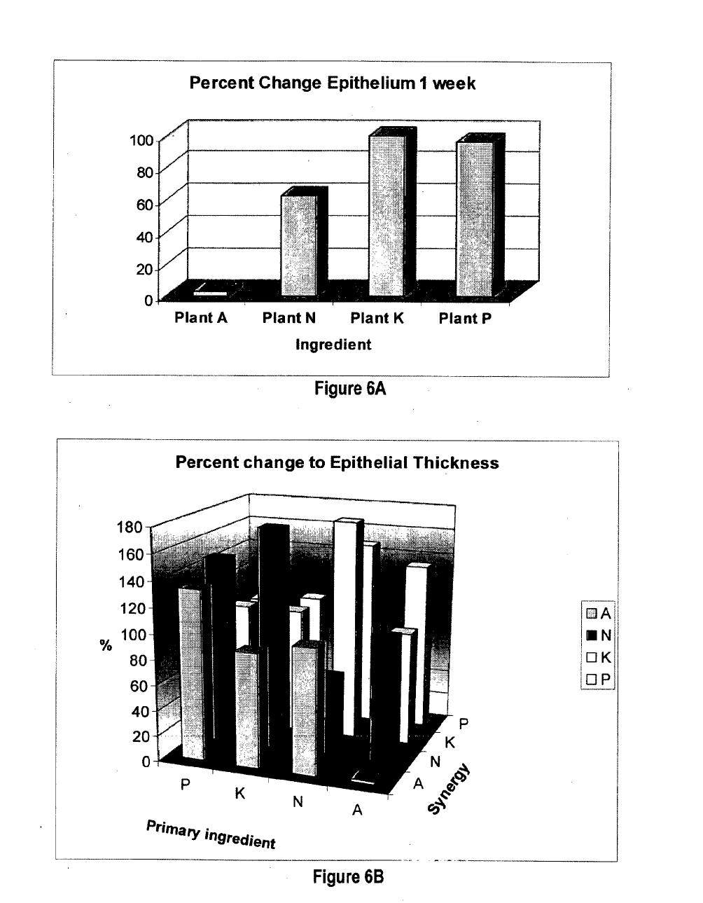

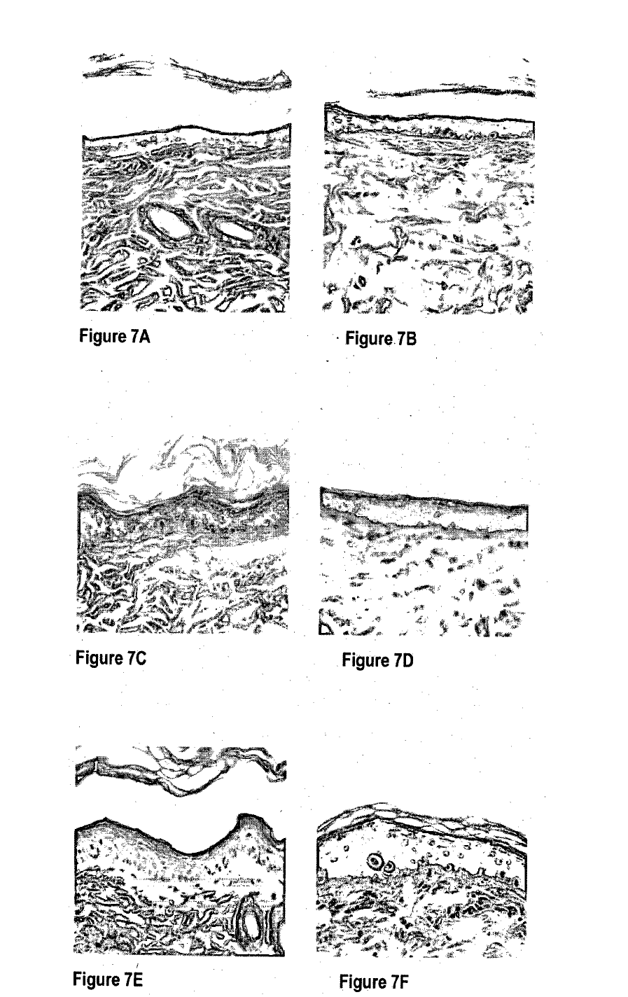

FIGS. 6A and 6B show the percentage change in the epithelium thickness at 7 days compared to control untreated skin on the same animal. FIG. 6A shows that topical application of Vigna marina (Burm.) Merr, extract in coconut oil and Terminalia catappa L. extract in coconut oil cause the largest increase in epithelium thickness over a 7 day period when the extract is applied alone. FIG. 6B details the percent change in epithelial thickness when each extract is applied in combination with one other extract. The results show that Cocos nucifera L. extract has a synergistic effect with both Vigna marina (Burm.) Merr. extract and Terminalia catappa L. extract and that Hibiscus tiliaceus L. has a synergistic effect with Vigna marina (Burm.) Merr.

### Example 9

### Preparation of Combination Extract of Vigna marina (Burm.) Merr., Cocos nucifera L., and Terminalia catappa L

The fresh crushed leaves, vine and bean of Vigna marina (Burm.) Merr. (2.5 kg), fresh husk of the young nut of Cocos nucifera L. (2.5 kg) and fresh crushed leaves, vine and bean of Terminalia catappa L (1 kg) were mulched separately.

Vigna marina (Burm.) Merr. was added to 95% ethanol (5 L) and agitated and the mixture allowed to sit for 12 h. The mulched material of Vigna marina (Burm.) Merr. was removed and added to a second vat of 95% ethanol (5 L) and the mixture allowed to sit for 12 h. The first 5 L of ethanol added to a large vat.

The mulched material of Vigna marina (Burm.) was removed from the second aliquot of ethanol and added to a third aliquot of 95% ethanol (5 L). The mulched material of Cocos nucifera L. was added to the second vessel of 95% ethanol (5 L) and allowed to stand for 12 h. The mulched material of Vigna marina (Burm.) (now almost colourless) was removed from the third aliquot of 95% ethanol and discarded. The mulched material of Cocos nucifera L. was removed from the second vessel and placed in the third aliquot of 95% ethanol. The third aliquot was allowed to stand for 12 h. The second aliquot of 95% ethanol was added to the first aliquot.

The mulched material of Cocos nucifera L. was removed from the third aliquot of 95% ethanol and placed into a fourth aliquot of 95% ethanol (5 L). The mulched material of Terminalia catappa L was added to third aliquot of 95% ethanol. Both the third and fourth aliquots were allowed to stand for 12 h.

The mulched material of Cocos nucifera L. was removed from the fourth aliquot and discarded. The mulched material of Terminalia catappa L was removed from the third aliquot of 95% ethanol and placed in the fourth aliquot of 95% ethanol and allowed to stand for 12 h. The third aliquot of 95% ethanol was added to the first and second aliquots.

The mulched material of Terminalia catappa L was removed from the fourth aliquot, added to a fifth aliquot of 95% ethanol (5 L) 95% ethanol and allowed to stand for 12 h. The mulched material of Terminalia catappa L (now colourless) was removed from the fifth aliquot. Both the fourth and fifth aliquots of 95% ethanol were added to the first three aliquots. To the resultant extracted mixture virgin coconut oil (1 L) was added. The mixture was mixed vigorously for 5 min every 15 min for a period of 4 h. The mixture was then heated to boiling and held at greater than 80° C. until the volume of the mixture was reduced from 26 L to 3 L. The resultant mixture was allowed to cool and settle. The coconut oil was decanted from the top of the mixture and placed in sealable vessels inverted in a refrigerator. The coconut oil was allowed to solidify and the remaining aqueous components removed and discarded. The coconut oil was then heated in a hot water bath at approximately 56° C. and filtered.

### Example 10

### Dose Dependence of Combination Extract of Vigna marina (Burm.) Merr., Cocos nucifera L., and Terminalia catappa L

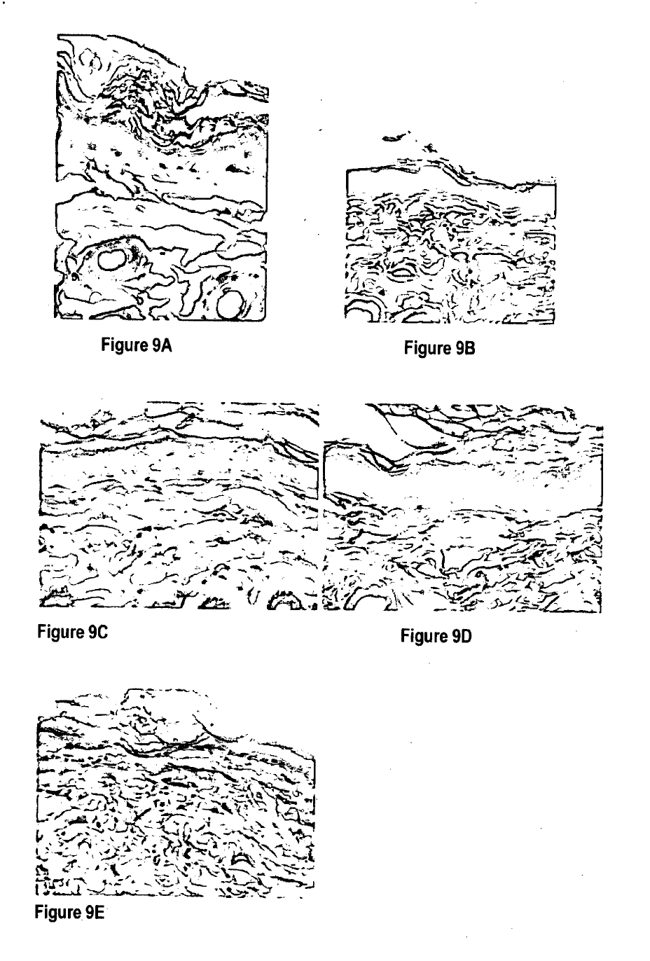

To assess the dose dependence of the combination extract prepared as described in Example 9, a daily dose 1 mL of the extract of was topically applied to the epithelial surface of the backs of 10-14 week old female rats. The concentration of the extract was 100%. The extract was also applied at 50%, 40%, 25%, 10%, 5% and 1%. These extracts were prepared by dilution of the 100% extract with coconut oil. The skin was treated with a daily topical application for 7 days. The control animals were untreated 10-14 week old female rats. FIGS. 9A to 9F show the histology of the skin at 7 days. The results demonstrate the dose dependent relationship of the extract with the epithelial thickest and the cellularity more apparent after a daily application of 50% or 100% of the extract.

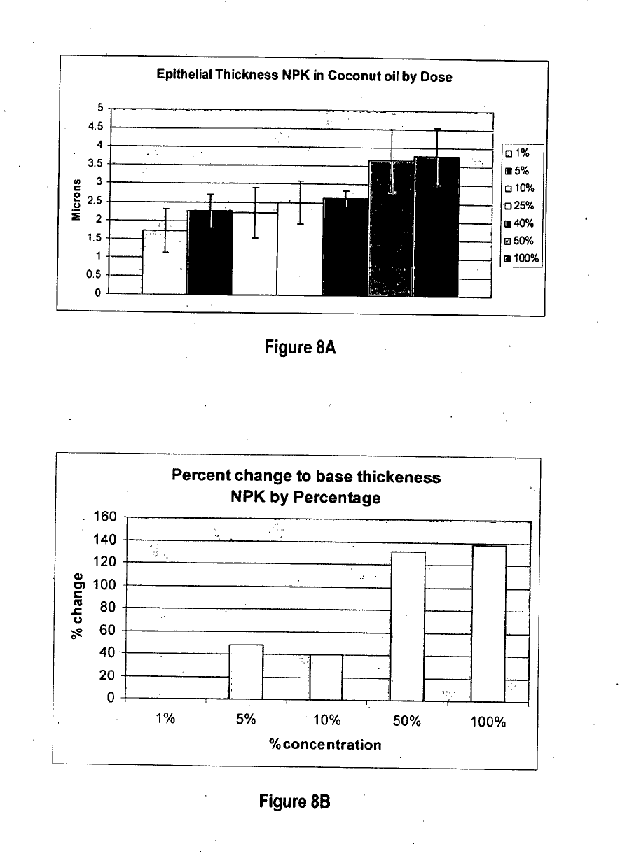

FIGS. 8A and 8B shows the epithelial thickness in microns and percent change in epithelial thickness of the skin at 7 days. An increase of over 130% in the epithelial thickness of the skin was observed for animals treated with 50% or 100% of the combination extract.

### Example 11

### Effect of Combination Extract of Vigna marina (Burm.) Merr., Cocos nucifera L., and Terminalia catappa L in Admixtures

The extract of Vigna marina (Burm.) Merr., Cocos nucifera L. and Terminalia catappa L. in coconut oil (100%) prepared as described in Example 9, was admixed with chlorhexidine; hydroderm moisturiser; 25% Chloramphenicol and 50% Chloramphenicol. Each mixture was applied (1 mL) topically to the epithelial surface of the backs of 10-14 week old female rats for 7 days. At 7 days the histology of the treated skin was assessed.

The histological profiles are shown in FIGS. 9A to 9E and show that the efficacy of the extract is not attenuated by the presence of commercial ointments or solutions and that the extract is compatible with antiseptic and antibiotic applications.

### Example 12

### Promotion of Wound Healing

A study was carried out to assess the wound healing capabilities of extracts of Vigna marina (Burm.) Merr., Cocos nucifera L., Terminalia catappa L. and Hibiscus tiliaceus L. prepared as described in Example 1. For the study 12 week old healthy female rats were used as an optimal healing model. Wounds were surgically created under anaesthesia and sterile conditions, and closed by primary intention (all tissues including the skin were closed with suture material) with intraoperative antibiotics and clean housing. A daily dose of 1 mL of the extract was applied topically to the surgical site. The extract was applied daily for as total of 10 days. The control animals received no treatment. At 3, 5, 7 and 10 days after creation of the site the wound histology was assessed and the wound tested for mechanical strength by distraction to failure testing.

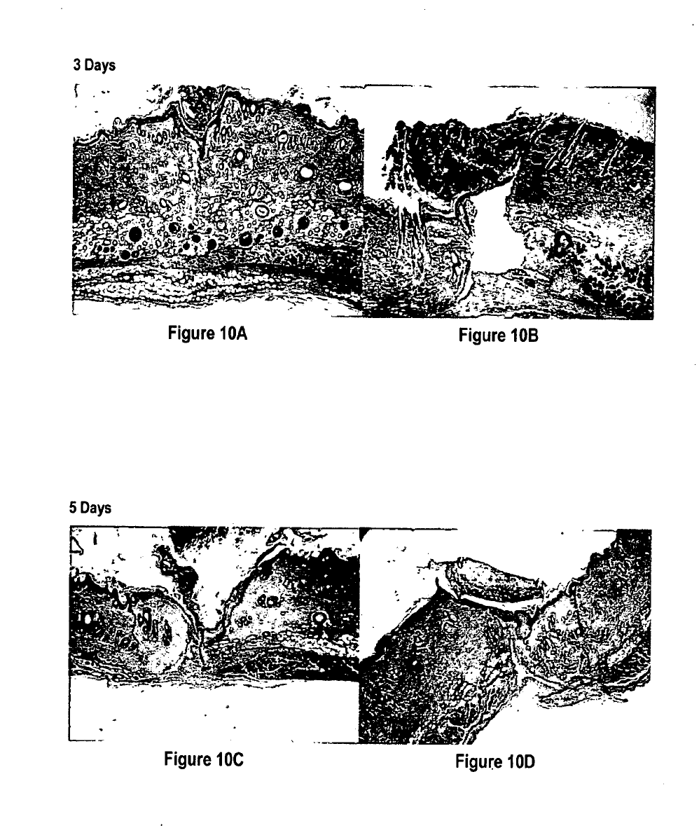

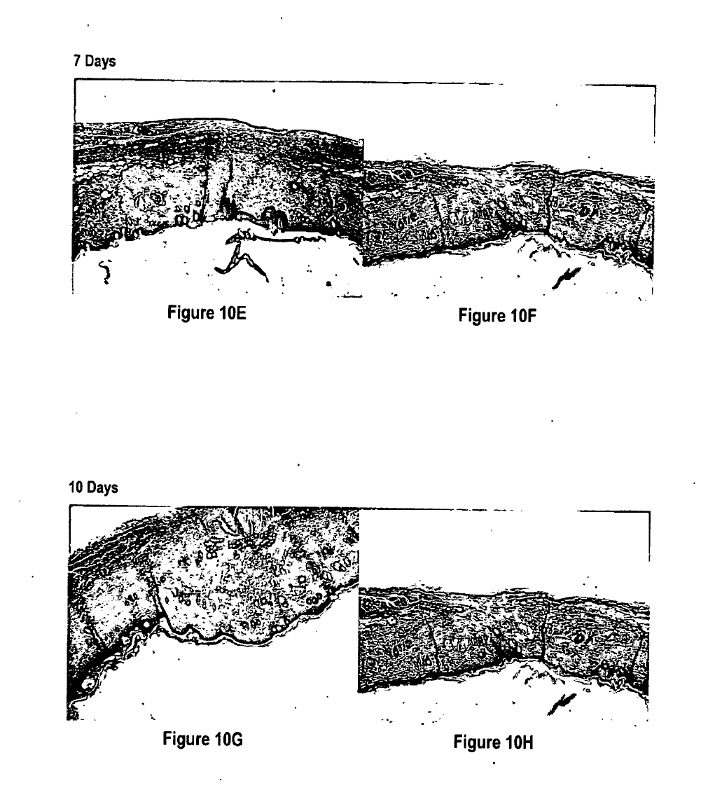

The wound histology of the untreated wounds (control) and treated wounds (test) are shown in FIGS. 10A to 10H. The histology of the treated wounds show an early proliferation of the epithelium covering the wound in the first 3 days, followed by an infiltration of cells into the wound to commence regeneration and complete healing by day 7 with minimal evidence of wound by day 10. Evidence of wound healing was also observed in the untreated wounds by day 10 but this occurred at a slower rate than was observed in the treated wounds, thereby demonstrating that the extract enhances the rate of the natural healing process.

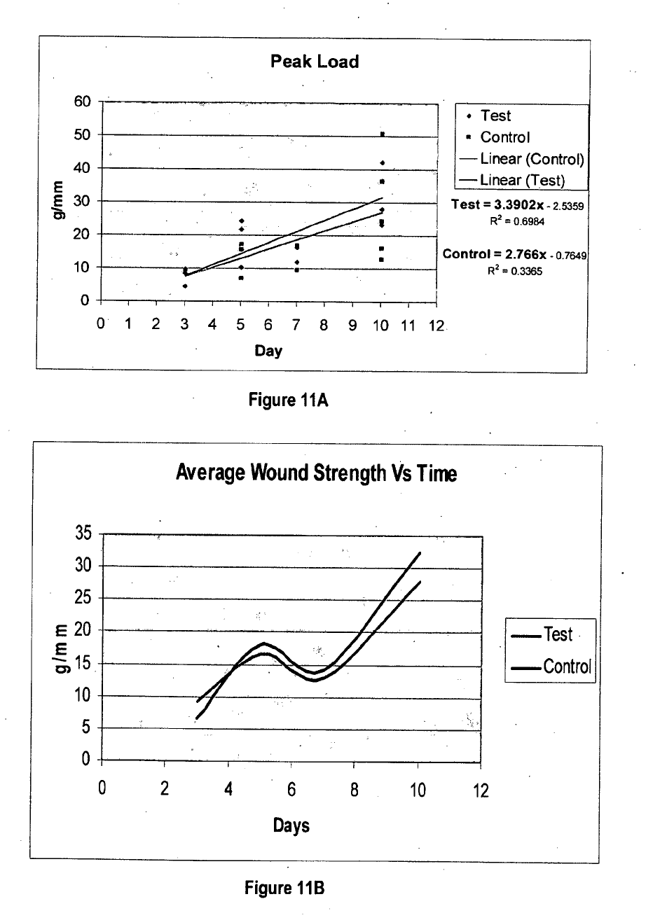

A graphical representation of the change in mechanical strength of the wound site during treatment is shown in FIG. 11A. The treated wound sites were found to have a 22% higher mechanical strength than the untreated wound sites. FIG. 11B shows that when the mechanical strength data was modelled into a non linear healing curve (to reflect healing pattern) the differences are preserved.

The results from the study show that the treatment of a wound with the combination extract of Example 1 both promotes wound healing and increases the mechanical strength of the skin at the wound site.

### Example 13

By way of example only suitable compositions for use in accordance with the invention are outlined below. The following are to be construed as merely illustrative examples of compositions and not as a limitation of the scope of the present invention in any way.

| W/O Cream | (%) |
|---|---|
| Paraffin | 10.00 |
| Ozokerite | 4.00 |
| Vaseline | 4.00 |
| Vegetable Oil | 10.00 |
| Wool Wax Alcohol | 2.00 |
| Aluminium Stearate | 0.40 |
| Vigna marina | 2.50 |
| Cocos nucifera | 5.00 |
| Preservative | q.s. |
| Fragrance | q.s. |
| Aqua | ad 100.00 |

| W/O Lotion | (%) |
|---|---|
| Paraffin | 25.00 |
| Silicon Oil | 2.00 |
| Ceresin | 1.50 |
| Wool Wax Alcohol | 0.50 |
| Glucose Sesqui Isostearate | 2.50 |
| Hibiscus tiliaceus | 0.60 |
| Terminalia catappa | 5.00 |
| Preservative | q.s. |
| Fragrance | q.s. |
| Aqua | ad 100.00 |

| O/W Lotion | (%) |
|---|---|
| Paraffin | 5.00 |
| Isopropyl Palmitate | 5.00 |
| Cetyl Alcohol | 2.00 |
| Bees Wax | 2.00 |
| Ceteareth-20 | 2.00 |
| PEG-20-Glyceryl Stearate | 1.50 |
| Glycerol | 3.00 |
| Cocos nucifera | 5.00 |
| Vigna marina | 2.00 |
| Preservative | q.s. |
| Fragrance | q.s. |
| Aqua | ad 100.00 |

| O/W Cream | (%) |
|---|---|
| Sunflower Oil | 7.50 |
| Cetyl Alcohol | 2.00 |
| Glyceryl Mono Stearate | 1.50 |
| PEG-30-Glyceryl Stearate | 2.00 |
| Glycerol | 3.00 |
| Isopropyl Palmitate | 5.00 |
| Carbopol 980 (neutralised) | 0.30 |
| Cocos nucifera | 7.50 |
| Terminalia catappa | 1.50 |
| Preservative | q.s. |
| Fragrance | q.s. |
| Aqua | ad 100.00 |

| Skin Balm | (%) |
|---|---|
| Vaseline | 36.00 |
| Ceresin | 10.00 |
| Zinc Oxide | 4.00 |
| Walnut Oil | 10.00 |
| Cocos nucifera | 10.00 |
| Terminalia catappa | 5.00 |
| Preservative | q.s. |
| Fragrance | q.s. |
| Paraffin | ad 100.00 |

| Skincare Oil | (%) |
|---|---|
| Cetyl Palmitate | 3.00 |
| C₁₂₋₁₅-Alkyl Benzoate | 2.00 |
| Poly iso-Butene | 10.00 |
| Squalane | 2.00 |
| Hibiscus tiliaceus | 0.20 |
| Cocos nucifera | 7.50 |
| Preservative | q.s. |
| Fragrance | q.s. |
| Paraffin | ad 100.00 |

| Bath Oil | (%) |
|---|---|
| Paraffin | 20.00 |
| PEG-40 Hydrogenated Castor Oil | 5.00 |
| Vigna marina | 2.50 |
| Hibiscus tiliaceus | 1.00 |
| Preservative | q.s. |
| Fragrance | q.s. |
| Soy Oil | ad 100.00 |

| Lip Stick | (%) |
|---|---|
| Ceresin | 8.00 |
| Bees Wax | 4.00 |
| Carnauba Wax | 2.00 |
| Vaseline | 40.00 |
| Hydrogenated Castor Oil | 4.00 |
| Caprylic/Capric Triglyceride | 6.00 |
| Cocos nucifera | 2.50 |
| Terminalia catappa | 2.50 |
| Preservative | q.s. |
| Fragrance | q.s. |
| Paraffin | ad 100.00 |

| Face Care Mask | (%) |
|---|---|
| PEG-50 Lanolin | 0.50 |
| Glyceryl Stearate | 2.00 |
| Sunflower Oil | 3.00 |
| Bentonite | 8.00 |
| Kaolin | 35.00 |
| Zinc Oxide | 5.00 |
| Ascorbyl Citrate | 0.05 |
| Hibiscus tiliaceus | 0.30 |
| Terminalia catappa | 2.50 |
| Preservative | q.s. |
| Fragrance | q.s. |
| Aqua | ad 100.00 |

| Liposomal Gel | (%) |
|---|---|
| Lecithin | 6.00 |
| Vegetable Oil | 10.00 |
| Hydrolysed Collagen | 2.00 |
| Xanthan Gum | 1.40 |
| Butylene Glycol | 3.00 |
| Cocos nucifera | 2.50 |
| Terminalia catappa | 0.70 |
| Arbutin | 0.10 |
| Preservative | q.s. |
| Fragrance | q.s. |
| Aqua | ad 100.00 |

| Shower Gel | (%) |
|---|---|
| Cocoamido Diacetate | 10.00 |
| Sodium Lauryl Sulfate | 25.00 |
| Potassium Cocoyl Hydrolysed Collagen | 5.00 |
| Macadamia Nut Oil | 5.00 |
| Sodium Chloride | 0.60 |
| Hibiscus tiliaceus | 0.10 |
| Vigna marina | 0.20 |
| Preservative | q.s. |
| Fragrance | q.s. |
| Aqua | ad 100.00 |

| Soap | (%) |
|---|---|
| Sodium Tallowate | 60.00 |
| Sodium Cocoate | 28.00 |
| Sodium Chloride | 0.50 |
| Vigna marina | 0.05 |
| Terminalia catappa | 0.20 |
| Preservative | q.s. |
| Fragrance | q.s. |
| Aqua | ad 100.00 |

| Syndet | (%) |
|---|---|
| Sodium Lauryl Sulfate | 30.00 |
| Sodium Sulfo Succinate | 10.00 |
| Potassium Cocoyl Hydrolysed Collagen | 2.00 |
| Dimethicon Copolyol | 2.00 |
| Paraffin | 2.00 |
| Corn Starch | 10.00 |
| Talcum | 10.00 |
| Glycerol | 3.00 |
| Terminalia catappa | 0.30 |
| Cocos nucifera | 0.50 |
| Preservative | q.s. |
| Fragrance | q.s. |
| Aqua | ad 100.00 |

| Hair Care Preparation | (%) |
|---|---|
| TEA-Cocoyl Hydrolysed Collagen | 30.00 |
| Mono Ethanolamine Lauryl Sulfate | 25.00 |
| Almond Oil | 2.00 |
| Sodium Chloride | 1.00 |
| Terminalia catappa | 2.50 |
| Cocos nucifera | 0.25 |
| Preservative | q.s. |
| Fragrance | q.s. |
| Aqua | ad 100.00 |

| Shampoo | (%) |
|---|---|
| Sodium Lauryl Sulfate | 34.00 |
| Disodium Lauryl Sulfosuccinate | 6.00 |
| Cocoamido Propyl Betaine | 10.00 |
| Glycol Distearate | 5.00 |
| Vigna marina | 0.30 |
| Terminalia catappa | 0.50 |
| Preservative | q.s. |
| Fragrance | q.s. |
| Aqua | ad 100.00 |

| Hair Care Stay-in Preparation | (%) |
|---|---|
| Cetyl Alcohol | 5.00 |
| Caprylic/Capric Triglyceride | 3.00 |
| Petrolatum | 2.00 |
| Wool Wax Alcohol | 0.50 |
| Hibiscus tiliaceus | 0.20 |
| Vigna marina | 0.90 |
| Preservative | q.s. |
| Fragrance | q.s. |
| Aqua | ad 100.00 |

| Conditioner | (%) |
|---|---|
| Cocoamido Propyl Betaine | 5.00 |
| Cetyl Alcohol | 2.00 |
| Propylene Glycol | 2.00 |
| Citric Acid | 0.30 |
| Hibiscus sabdariffa | 0.50 |
| Terminalia catappa | 2.50 |
| Preservative | q.s. |
| Fragrance | q.s. |
| Aqua | ad 100.00 |

| Setting Lotion | (%) |
|---|---|
| Polyvinyl Pyrrolidone/Vinyl Acetate/ | 5.00 |
| Vinyl Propionate Copolymer | |
| Ethanol | 45.00 |
| Terminalia ferdinandiana | 0.20 |
| Cocos nucifera | 0.50 |
| Preservative | q.s. |
| Fragrance | q.s. |
| Aqua | ad 100.00 |

| Styling Cream | (%) |
|---|---|
| Vaseline | 4.00 |
| Cetearyl Alcohol | 4.00 |
| PEG-40 Hydrogenated Castor Oil | 2.00 |
| Isopropyl Palmitate | 5.00 |
| Citric Acid | 1.00 |
| Terminalia catappa | 0.70 |
| Cocos nucifera | 0.20 |
| Preservative | q.s. |
| Fragrance | q.s. |
| Aqua | ad 100.00 |

| Shaving Foam | (%) |
|---|---|
| Stearic Acid | 7.00 |
| Sodium Lauryl Sulfate | 3.00 |
| Stearyl Alcohol | 1.00 |
| Glycerol | 5.00 |
| Triethanol Amine | 3.60 |
| Cocos nucifera | 2.50 |
| Terminalia catappa | 0.50 |
| Preservative | q.s. |
| Fragrance | q.s. |
| Aqua | ad 100.00 |

| Foot Cream | (%) |
|---|---|
| Soluan 5 | 2.00 |
| Methyl Salicylate | 5.00 |
| Caprylic/Capric Triglyceride | 10.00 |
| Stearic Acid | 5.00 |
| Cetyl Alcohol | 1.00 |
| Glycerol | 2.00 |
| Dimethicon | 1.00 |
| Carbopol 984 | 0.50 |
| Triethanol Amine | 1.50 |
| Terminalia ferdinandiana | 2.50 |
| Hibiscus sabdariffa | 2.50 |
| Preservative | q.s. |
| Fragrance | 0.40 |
| Aqua | ad 100.00 |

| Deodorant Aerosol Spray | (%) |
|---|---|
| Octyl Dodecanol | 0.50 |
| Terminalia catappa | 0.50 |
| Vigna marina | 0.50 |
| Preservative | q.s. |
| Fragrance | q.s. |
| Ethanol | ad 100.00 |

The resulting liquid phase is filled together with a propane/butane mix (3:7) at a ratio of 39:61 into aerosol cans.

| Microemulsion | (%) |
|---|---|
| 1.3-Di-(2-ethylhexyl)-cyclohexane | 35.00 |
| Glyceryl Lanolate | 5.00 |
| Sorbitan Monolaurate | 10.00 |
| Citric Acid | q.s. |
| Terminalia catappa | 0.40 |
| Cocos nucifera | 0.50 |
| Preservative | q.s. |
| Fragrance | q.s. |
| Aqua | ad 100.0 |

| Gel Toothpaste | (%) |
|---|---|
| Sodium Carboxy Methyl Cellulose | 0.40 |
| Sorbitol 70% | 72.00 |
| PEG 1500 | 3.00 |
| Sodium Saccharinate | 0.07 |
| Sodium Fluoride | 0.24 |
| PHB Ethyl Ester | 0.15 |
| Flavour | 1.00 |
| β-D-Octyl Gluco Pyranoside | 3.00 |
| Abrasive Silica | 11.00 |
| Thickening Silica | 6.00 |
| Sodium Lauryl Sulfate | 1.40 |
| Terminalia catappa | 0.30 |
| Cocos nucifera | 2.50 |
| Preservative | q.s. |
| Aqua | Ad 100.00 |

| Mouthwash | (%) |
|---|---|
| Ethanol 94.7% | 7.00 |
| Glycerol 86% | 12.00 |
| Sodium Fluoride | 0.05 |
| Pluronic F-127 | 1.40 |
| Sodium Phosphate Buffer pH 7.0 | 1.10 |
| Sorbic Acid | 0.20 |
| Sodium Saccharinate | 0.10 |
| Flavour | 0.15 |
| β-D-Octyl Gluco Pyranoside | 0.40 |
| Colourant | 0.01 |
| Terminalia catappa | 0.50 |
| Cocos nucifera | 0.30 |
| Preservative | q.s. |
| Aqua | Ad 100.00 |

The claims defining the invention are as follows:

1. A composition comprising a biologically active extract of one or more of Vigna sp., Terminalia sp. or Cocos nucifera for maintaining and improving the health and/or appearance of the skin and/or an epidermal appendage.

2. A composition of claim 1 further comprising a biologically active extract of Hibiscus sp.

3. A composition of claim 1 or claim 2, wherein the improvement comprises a reduction in the appearance of fine line and wrinkles, an increase in skin radiance, an increase in skin tone, an increase in skin texture and/or firmness, a reduction in acne scars, a decrease in age spots, a reduction in the blotchiness, broken capillaries, intracutaneous and/or subcutaneous bleeding of the skin, an increase in the lightness of skin and/or an epidermal appendage, a reduction of visible pores, a reduction in stretch marks, an increase in skin elasticity and/or a reduction in skin sagging, a reduction in skin roughness, a reduction in skin sensitivity, restoration of the physiological balance of the skin and/or an epidermal appendage, an increase in trans-epidermal water loss, or an increase in the natural moisturising factors in the skin.

4. A composition of any one of claims 1 to 3, wherein the composition is applied to aging or aged skin.

5. A composition of claim 4, wherein the aging or aged skin has age related skin atrophy such as associated with oestrogen deficiency.

6. A composition of any one of claims 1 to 5, wherein the Vigna sp. is Vigna marina (Burm.) Merr.

7. A composition of any one of claims 1 to 6, wherein the Terminalia sp. is Terminalia catappa L.

8. A composition of any one of claims 2 to 7, wherein the Hibiscus sp. is Hibiscus tiliaceus L.

9. A composition of any one of claims 1 to 5, wherein the extract comprises a combination of an extract of Vigna marina (Burm.) Merr. and Terminalia catappa L.

10. A composition of any one of claims 1 to 5, wherein the extract comprises a combination of an extract of Vigna marina (Burm.) Merr. and Cocos nucifera L.

11. A composition of any one of claims 1 to 5, wherein the extract comprises a combination of an extract of Vigna marina (Burm.) Merr. and Cocos nucifera L., and Terminalia catappa L

12. A composition of any one of claims 2 to 8, wherein the extract comprises a combination of an extract of Vigna marina (Burm.) Merr., Cocos nucifera L., Terminalia catappa L. and Hibiscus tiliaceus L.

13. A composition of any one of claims 1 to 12, wherein the extract is derived from one or more of a leaf, root, bark, wood, stem, fruit, shoot, seed, seed capsule, vine, bean, flower, husk or nut.

14. A composition of any one of claims 1 to 13, wherein the Vigna marina (Burm.) Merr., Terminalia catappa L., and/or Cocos nucifera extract is derived from one or more of a leaf, vine, husk and/or bean.

15. A composition of any one of claims 2 to 13, wherein the Hibiscus tiliaceus L. extract is derived from one or more of a leaf, vine and/or bean.

16. A composition of any, one of claims 1 to 15, wherein the extract is prepared using and/or comprises, a lipid, a plant based oil, a hydrocarbon and/or an alcohol.

17. A composition of claim 16, wherein the plant based oil is derived from seeds or fruit.

18. A composition of claim 16 or claim 17, wherein the plant based oil is Cocos nucifera oil.

19. A composition of claim 18, wherein the Cocos nucifera oil is virgin Cocos nucifera oil, refined Cocos nucifera oil, hydrogenated Cocos nucifera oil or fractionated Cocos nucifera oil.

20. A composition of any one of claims 1 to 19, further comprising a cosmetically or pharmaceutically acceptable carrier, diluent and/or excipient.

21. A composition of any one of claims 1 to 20, wherein the composition is applied topically to the skin or to an epidermal appendage of a subject.

22. A composition or claim 21, wherein the epidermal appendage is hair, hair follicles, nails, cuticles, or teeth.

23. A method for maintaining and improving the health and/or appearance of the skin and/or an epidermal appendage, the method comprising applying to the skin and/or an epidermal appendage of a subject a composition comprising a biologically active extract of one or more of Vigna sp., Terminalia sp. or Cocos nucifera.

24. A method of claim 23 further comprising a biologically active extract of Hibiscus sp.

25. A method of claim 23 or claim 24, wherein the improvement comprises a reduction in the appearance of fine line and wrinkles, an increase in skin radiance, an increase in skin tone, an increase in skin texture and/or firmness, a reduction in acne scars, a decrease in age spots, a reduction in the blotchiness, broken capillaries, intracutaneous and/or subcutaneous bleeding of the skin, an increase in the lightness of skin and/or an epidermal appendage, a reduction of visible pores, a reduction in stretch marks, an increase in skin elasticity and/or a reduction in skin sagging, a reduction in skin roughness, a reduction in skin sensitivity, restoration of the physiological balance of the skin and/or an epidermal appendage, an increase in trans-epidermal water loss, or an increase in the natural moisturising factors in the skin.

26. A method of any one of claims 23 to 25, wherein the composition is applied to aging or aged skin.

27. A method of claim 26, wherein the aging or aged skin has age related skin atrophy such as associated with oestrogen deficiency.

28. A method of any one of claims 23 to 27, wherein the Vigna sp. is Vigna marina (Burm.) Merr.

29. A method of any one of claims 23 to 28, wherein the Terminalia sp. is Terminalia catappa L.

30. A method of any one of claims 24 to 29, wherein the Hibiscus sp. is Hibiscus tiliaceus L.

31. A method of any one of claims 23 to 30, wherein Cocos nucifera oil is applied to the skin and/or epidermal appendage prior to or after the composition is applied.

32. A method of any one of claims 23 to 31, wherein the subject is a human.

33. Use of a composition comprising a biologically active extract of one or more of Vigna sp., Terminalia sp. or Cocos nucifera for the manufacture of a medicament for maintaining and improving the health and/or appearance of the skin and/or an epidermal appendage.

34. A use of claim 33 further comprising a biologically active extract of Hibiscus sp.

35. A use of claim 33 or claim 34, wherein the improvement comprises a reduction in the appearance of fine line and wrinkles, an increase in skin radiance, an increase in skin tone, an increase in skin texture and/or firmness, a reduction in acne scars, a decrease in age spots, a reduction in the blotchiness, broken capillaries, intracutaneous and/or subcutaneous bleeding of the skin, an increase in the lightness of skin and/or an epidermal appendage, a reduction of visible pores, a reduction in stretch marks, an increase in skin elasticity and/or a reduction in skin sagging, a reduction in skin roughness, a reduction in skin sensitivity, restoration of the physiological balance of the skin and/or an epidermal appendage, an increase in trans-epidermal water loss, or an increase in the natural moisturising factors in the skin.

36. A use of any one of claims 33 to 35, wherein the composition is applied to aging or aged skin.

37. A use of claim 36, wherein the aging or aged skin has age related skin atrophy such as associated with oestrogen deficiency.

38. A use of any one of claims 33 to 37, wherein the Vigna sp. is Vigna marina (Burm.) Merr.

39. A use of any one of claims 33 to 38, wherein the Terminalia sp. is Terminalia catappa L.

40. A use of any one of claims 34 to 39, wherein the Hibiscus sp. is Hibiscus tiliaceus L.
